(19) United States
(12) Patent Application Publication
(10) Pub. No.: US 2025/0195298 A1
(43) Pub. Date: Jun. 19, 2025

(54) ABSORBENT ARTICLE

(71) Applicant: The Procter & Gamble Company, Cincinnati, OH (US)

(72) Inventors: Ernesto Gabriel BIANCHI, Oberursel (DE); Jacob Alan BARNHORST, Deerfield Township, OH (US); Hossein SHAKAROUN, Frankfurt (DE); Ute FROEHLICH, Frankfurt (DE)

(21) Appl. No.: 18/976,779

(22) Filed: Dec. 11, 2024

Related U.S. Application Data
(60) Provisional application No. 63/608,327, filed on Dec. 11, 2023.

(51) Int. Cl. A61F 13/537; A61F 13/53; A61F 13/534; A61F 13/539

(52) U.S. Cl. CPC A61F 13/53708; A61F 13/539; A61F 2013/530489; A61F 2013/530496; A61F 2013/5349; A61F 2013/53908

(57) ABSTRACT

A personal hygiene absorbent article is disclosed. The personal hygiene absorbent article includes an absorbent layer having a narrow portion between the front edge and the back edge of the absorbent layer, and a distribution layer having a narrow portion between the front edge and the back edge of the distribution layer.

### CROSS REFERENCE TO RELATED APPLICATION

This application claims the benefit, under 35 U.S.C. § 119(e), of U.S. Provisional Application No. 63/608,327, filed Dec. 11, 2023, the substance of which is incorporated herein by reference.

### FIELD OF THE INVENTION

The invention relates to personal hygiene absorbent articles, such as baby diapers and adult incontinence products.

### BACKGROUND OF THE INVENTION

Modern personal hygiene absorbent articles are an assembly of different layers. Baby and adult diapers typically comprise a fluid-permeable topsheet on the wearer-facing side of the article, a liquid management system underneath the topsheet, an absorbent core, and a fluid-impermeable backsheet on the garment-facing side of the article. The liquid management system can quickly acquire and distribute the liquid away from the topsheet into the absorbent core. The liquid management system may typically consist of an acquisition layer, or an assembly consisting of an acquisition layer and a distribution layer. The absorbent core typically comprises an absorbent material disposed in a core wrap. The absorbent material typically comprises superabsorbent polymer particles (“SAP”) blended with cellulose fibers. Over the years, the relative SAP content has increased and the cellulose fibers reduced, thus providing thinner absorbent cores. Absorbent cores without cellulose fibers, so called airfelt-free cores, have been more recently used.

While leakage prevention is primarily provided by the permanent immobilization of urine in the absorbent core, modern personal hygiene absorbent articles comprise other components, in particular a gasketing system, to further improve the performance of the articles. Gasketing systems typically comprise a pair of inner leg cuffs and a pair of outer leg cuffs that help containing the fluid near the body discharge area. The inner leg cuffs (also referred to as barrier leg cuffs) have a proximal edge joined to the chassis of the article and an elasticized distal edge that can stand up from the chassis to fit tight against the wearer's skin. The outer leg cuffs (also referred to as gasketing cuffs) are also elasticized but are formed in the same plane as the chassis of the articles. Such gasketing systems improve the fit of the article and provide a temporary storage for urine until the fluid is absorbed by the absorbent material.

Absorbent cores can absorb many times their weight of urine. One of the key limiting factor is the kinetic of absorption. Absorbent articles tend to sag down in the crotch region due to the weight of the fluid absorbed. Sagging may cause the inner or outer leg cuffs to lose contact along the thighs of the wearer, thus increasing the possibility of leakages. While the leg cuffs normally maintain contact and fit, efficiency is limited for heavily loaded diapers especially if the diaper was not put in place correctly or was displaced out of position by the wearer. Sagging is a common cause for side leakages of absorbent articles such as baby diapers.

Increasing the height and width of the inner and outer cuffs respectively may reduce this problem, however this requires wider chassis designs at crotch and the additional materials can negatively impact fit and cost.

Despite the improvements suggested in the prior art, there is a continuous need for improving dry and wet fit, wearing comfort, ease of application and fluid handling properties, in particular reducing leakage especially at the legs, of absorbent articles while keeping the cost of production as low as possible. The present invention addresses these multiple requirements.

### SUMMARY OF THE INVENTION

The present invention is for a personal hygiene absorbent article. The article comprises a longitudinal axis extending in a longitudinal direction, a transversal axis extending in a transversal direction, and extends in a vertical direction orthogonal to the transversal and longitudinal direction. The article comprises in this vertical order: a liquid permeable topsheet, optionally an acquisition layer, a fibrous distribution layer, an absorbent core and a liquid-impermeable backsheet.

The absorbent core comprises a core wrap having a top layer and a bottom layer, and an absorbent layer comprising superabsorbent polymer particles and optionally cellulose fibers. The absorbent layer is disposed between the core wrap top layer and the core wrap bottom layer. The absorbent layer, when seen in the plane formed by the longitudinal and transversal directions, has a front edge, a back edge and two longitudinal edges joining the front and back edges. The absorbent layer has a maximum width W1, the width being measured in the transversal direction, and a length L′ measured in the longitudinal direction along the longitudinal axis. The absorbent layer comprises a narrow portion disposed intermediate the front edge and the back edge of the absorbent layer, the narrow portion having a minimum width W2. According to the invention, the absorbent layer's ratio of the maximum width W1 to the minimum width W2 is least 1.1 (W1≥1.1×W2).

The fibrous distribution layer has a front edge, a back edge, and two longitudinal edges joining the front edge and back edge. The distribution layer has a maximum width W3. The distribution layer comprises a narrow portion between the front edge and the back edge of the distribution layer, the narrow portion having a minimum width W4. The ratio of the maximum width W3 to the minimum width W4 of the distribution layer is at least 1.1 (W3≥1.1×W4).

The narrow portion of the absorbent layer and the narrow portion of the distribution layer are at least partially superposed. Furthermore, W1 is larger than or equal to W3 and W2 is larger than or equal to W4.

At least one of the absorbent layer and the distribution layer comprises at least one longitudinally-extending core channel and/or longitudinally-extending distribution channel respectively.

The absorbent core may comprise one or more such core channel(s). A core channel is an area within the absorbent material layer that is substantially free of absorbent material and can distribute fluid in the longitudinal direction. The core channel(s) is/are at least partially comprised in the narrow portion of the absorbent layer. The top layer of the core wrap is preferably bonded to the bottom layer of the core wrap through the core channels(s) to increase the longevity of the channel(s). When the absorbent material surrounding the core channel(s) absorbs a fluid such as urine and swells, a three-dimensional depression is formed along the length of the bonded core channel(s).

The fibrous distribution layer may comprise one or more distribution channel(s), which are area(s) free of distribution-material within the distribution layer. The distribution channel(s) are advantageously vertically superposed with the channel(s) of the absorbent core. The distribution channel(s) may also be shorter than the channel(s). The distribution layer may comprise or consists of cross-linked cellulose fibers.

The absorbent layer may be free of cellulosic fibers. The absorbent material may consist of superabsorbent polymer particles immobilized by a microfibrous glue.

The absorbent article may advantageously comprise an elasticized gasketing system.

Each of the above features may be combined in an inventive article according to the invention. The absorbent article may be in the form of a taped diaper (for baby, child or adult incontinence), a pant diaper (for baby, child or adult incontinence) or an insert to be placed in an underwear-like cover material.

### BRIEF DESCRIPTION OF THE DRAWINGS

FIG. 1 is a top view of the wearer-facing side of an exemplary taped diaper according to the invention which has been pulled flat, with some layers partially removed;

FIG. 2 shows a partially exploded view of the taped diaper of FIG. 1;

FIG. 3 is a schematic top view of the absorbent core of the taped diaper of FIG. 1 with the top layer partially removed;

FIG. 4 shows a partially exploded view of the absorbent core of FIG. 3;

FIG. 5A and FIG. 5B are schematic depiction of a cross-section of the core in dry and wet state respectively;

FIG. 6 shows a fibrous distribution layer disposed on an acquisition layer;

FIG. 7 shows a partially exploded view of an alternative taped diaper with core channels but without distribution channels;

FIG. 8 shows a partially exploded view of an alternative taped diaper with distribution channels but without core channels;

FIGS. 9-11 show alternative distribution layer that may be used with or without an acquisition layer;

FIGS. 12-13 show schematic top views of alternative absorbent cores; and

FIGS. 14-15 show additional schematic top views of alternative absorbent cores.

### DETAILED DESCRIPTION OF THE INVENTION

### Introduction

Any preferred, advantageous or example forms described below are not limiting the scope of the claims, unless specifically indicated to do so. The words “typically”, “normally”, “preferably”, “advantageously”, “in particular” and the likes also qualify features which are not intended to limit the scope of the claims, unless specifically indicated to do so. Any feature or component described herein in relation with one embodiment may be combined with another feature or component of another embodiment unless indicated otherwise.

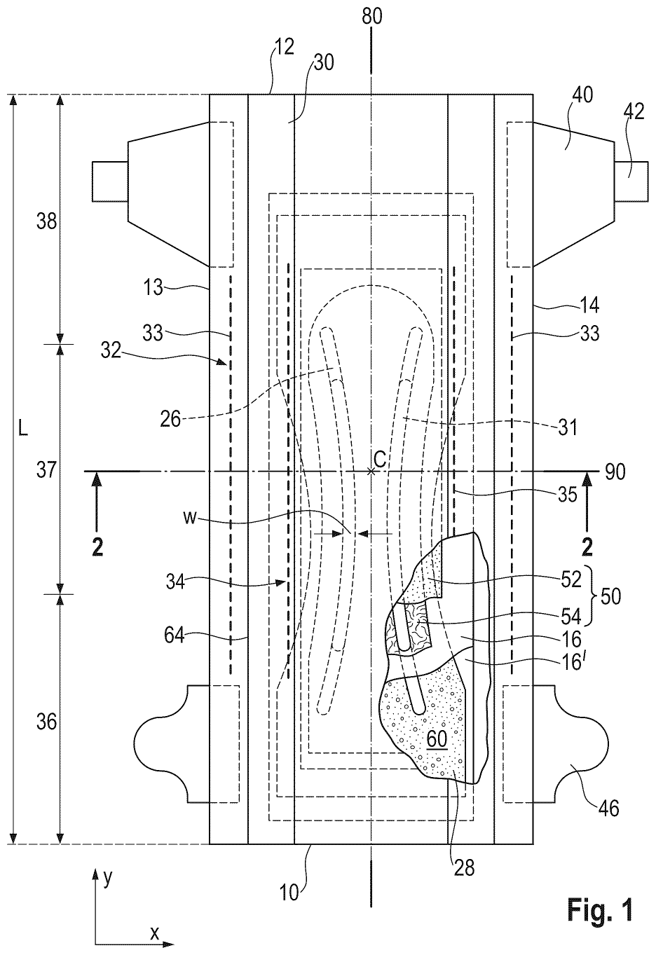

Unless indicated otherwise, the description and claims refer to the absorbent article, absorbent core or component thereof before use (i.e. dry, and not loaded with a fluid) and conditioned at least 24 hours at 21° C.+/−2° C. and 50+/−20% Relative Humidity (RH) and in a flat state as shown for example on FIG. 1.

A “nonwoven web” or “nonwoven” as used herein means a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yarn). Nonwoven webs can be formed by many processes such as meltblowing, spunbonding, solvent spinning, electrospinning, carding and airlaying. The basis weight of nonwoven webs is usually expressed in grams per square meter (g/m2 or gsm).

The absorbent articles of the invention and their components will now be discussed generally and with exemplary reference to the Figures and the numerals referred to in these Figures for illustration purpose. These examples are not intended to limit the scope of the claims unless specifically indicated.

### General Description of an Absorbent Article 20

Personal hygiene absorbent articles according to the invention are intended to be placed in the crotch portion of an incontinent wearer to absorb body exudates such a urine. The absorbent articles according to the invention include in particular baby and toddler diapers (including training pants), feminine sanitary pads and adult incontinence articles.

An exemplary absorbent article according to the invention is represented in FIG. 1. This diaper is shown for illustration purpose in the form of a taped diaper 20. FIG. 1 is a top plan view of the exemplary diaper 20 in a flat-out state with portions of the structure being cut-away to show the construction of the diaper more clearly. The absorbent article of the invention may have other forms, not represented, for example a pant-type article with pre-formed side seams. Unless otherwise indicated, dimensions and areas disclosed herein apply to the article in this flat-out configuration. Any width and length are measured respectively parallel to the transversal direction and longitudinal direction, as defined below. If some part of the article is under tension due to elasticized components, the article may be typically flattened using clamps along the periphery of the article and/or a sticky surface, so that the topsheet and backsheet can be pulled taut to be substantially flat. Closed articles such as diaper pants may be cut open along the side seams to apply them on a flat surface.

The absorbent article 20 comprises a front edge 10, a back edge 12, and two longitudinal side (i.e. longitudinally-extending lateral) edges 13, 14 joining the front edge and the back edge. The front edge 10 is the edge of the article which is intended to be placed towards the front of the user when worn, and the back edge 12 is the opposite edge. The absorbent article is notionally divided by a longitudinal axis 80 extending from the front edge to the back edge of the article and dividing the article in two substantially symmetrical halves relative to this axis, when viewing the article from the wearer facing side in a flat-out configuration, as exemplarily shown in FIG. 1. The longitudinal axis 80 may typically be aligned with the longitudinal axis 80′ of the absorbent core 28. The article has a length L as measured along the axis 80 from the back edge to the front edge. The absorbent article 20 can also be notionally divided by a transversal axis 90 into a front region and a back region of equal length measured on the longitudinal axis, when the article is in such a flat state. This article's transversal axis 90 is perpendicular to the longitudinal axis 80 and placed at half the length L of the article. The intersection of the longitudinal axis and transversal axis are referred herein as the Crotch Point “C”.

The absorbent article is further notionally divided in a front region 36, a back region 38 and a crotch region 37 in-between. The front region 36 is defined as the region of the article extending from the front edge 10 and having a length of a third of L along the longitudinal axis 80. The back region 38 is defined as the region of article extending from the back edge 12 of the article and having a length of one third of L along the longitudinal axis 80. The crotch region 37 is the intermediate region between the front and back regions, and also having a length of a third of L as measured along the longitudinal axis 80.

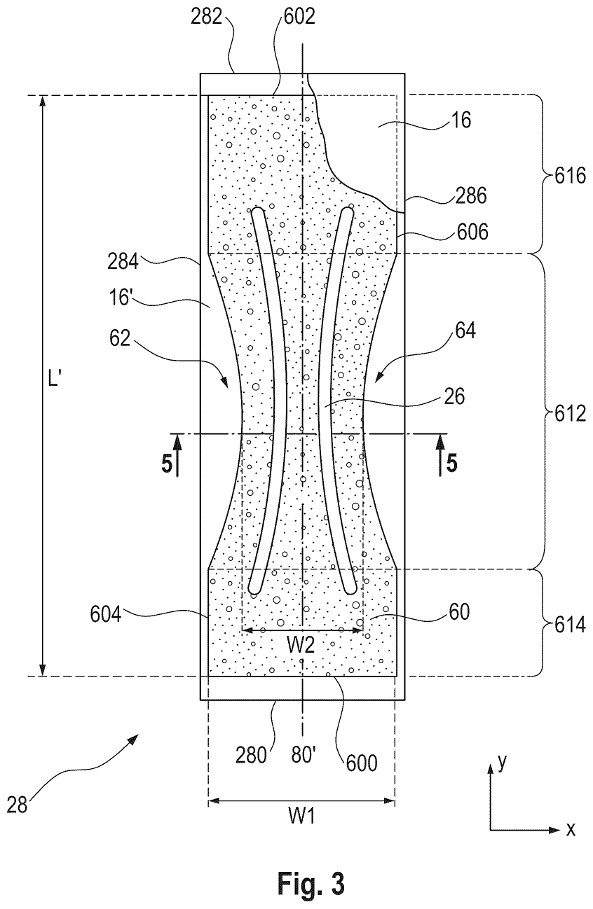

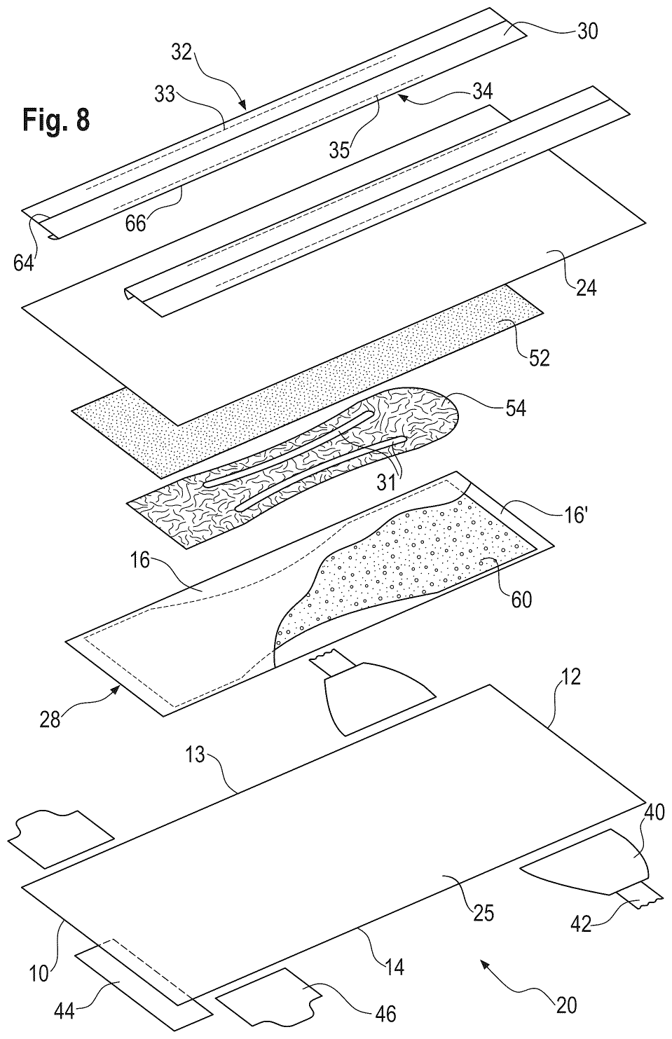

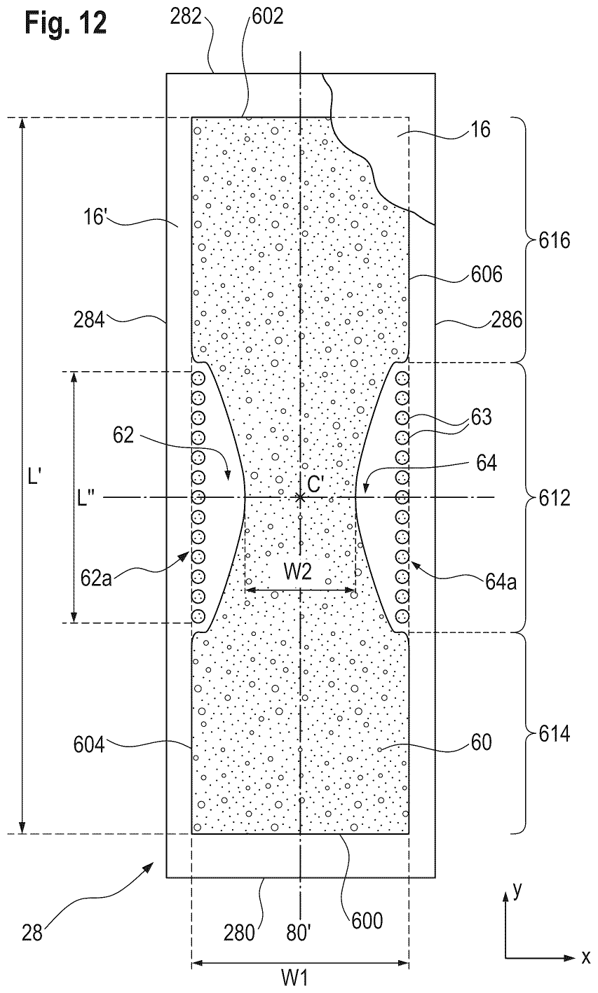

The absorbent article 20 comprises a wearer-facing side, which may be principally formed by a liquid permeable topsheet 24, a garment-facing surface which may be formed by a liquid impermeable backsheet 25, and an absorbent core 28 between the topsheet 24 and the backsheet 25. The absorbent core 28 is shown in isolation in FIG. 3 with channels. Alternative absorbent cores may be used, for example without core channels as illustrated in FIGS. 8 and 12. The topsheet 24, the backsheet 25, the absorbent core 28 and the other article components may be assembled in a variety of well-known configurations, in particular by gluing and/or heat embossing. Exemplary diaper assemblies are for example generally described in U.S. Pat. Nos. 3,860,003, 5,221,274, 5,554,145, 5,569,234, 5,580,411, and 6,004,266.

The absorbent article is preferably thin. In particular, the absorbent article may have a caliper of from 2.0 mm to 8.0 mm, or from 3.0 mm to 6.0 mm, measured at the crotch point C using the Thickness Measurement Method described below.

### General Description of the Absorbent Core 28

As used herein, the term “absorbent core” refers to an absorbent material layer (herein “absorbent layer”) disposed in a core wrap. The core wrap material may be a nonwoven or tissue paper material defining a top layer and bottom layer. The core wrap may be sealed transversally and/or and longitudinally.

The term “absorbent core” as used herein does not include a separate liquid management layer that temporarily acquire and distribute the fluid, in particular the distribution layer and the acquisition layer if present. The absorbent core is typically the component of an absorbent article that has the most absorbent capacity of all the components of the absorbent article, and which comprises all, or at least the majority of, superabsorbent polymer (SAP). The core may consist essentially of, or consist of, the core wrap, the absorbent material and optionally adhesives. The terms “absorbent core” and “core” are used interchangeably herein. The absorbent core is typically an individual component, which is converted with other components such as a topsheet and a backsheet to form the absorbent article in a converting line. However it is not excluded that the absorbent core may be formed directly on the backsheet, the topsheet or another layer, in which case the core wrap may be at least partially formed by one of these layers.

The absorbent cores of the invention are typically substantially planar. By substantially planar, it is meant that the absorbent core can be laid flat on a planar surface, as represented in FIG. 3 and that the transversal and longitudinal dimensions of the core are much larger than the vertical dimensions (at least before use). The absorbent cores may also be typically thin and conformable, so that they can also be laid on a curved surface for example a drum during the making process, or stored and handled as a continuous roll of stock material before being converted into an absorbent article. The thickness of the absorbent core (before use) as measured in the vertical direction is relatively small compared to dimensions in the transversal direction (x) and the longitudinal direction (y). These directions also correspond respectively to the transversal direction and longitudinal direction respectively of the article in which the absorbent core is integrated. Unless otherwise indicated, dimensions and areas disclosed herein apply and are measured on the core in this flat-out configuration as shown in FIG. 3. The same applies to an absorbent article, as exemplarily represented in FIG. 1 as a taped diaper, in which the absorbent core is integrated.

The absorbent cores and articles of the invention are now discussed with reference to the Figures and the numerals referred to in these Figures. These examples are not intended to limit the scope of the claims unless specifically indicated.

As illustrated in FIGS. 3-5, the absorbent core 28 comprises a front edge 280, a back edge 282 and two longitudinal edges 284, 286 joining the front edge and the back edge. The front edge of the core is intended to be placed towards the front of the absorbent article in which the core is or will be integrated. The front edge 280 and the back edge 282 of the core are typically shorter than the longitudinal edges 284, 286 of the core. The absorbent core can notionally comprise a longitudinal axis 80′ parallel to the longitudinal direction y and extending from the front edge 280 to the back edge 282, and dividing the core in two substantially symmetrical halves relative to this axis.

The absorbent core also comprises a top layer 16 and a bottom layer 16′ which form the core wrap. The top side of the core is placed towards the topsheet of the absorbent article and the bottom side is the side placed towards the backsheet 25 of the absorbent article. Typical core wrap materials are nonwovens, paper, tissues, films, ovens, or laminate of any of these. The core wrap material may in particular be a nonwoven, such as a carded nonwoven, a spunbond nonwoven (“S”) or a meltblown nonwoven (“M”), and laminates of any of these. For example, spunmelt polypropylene nonwovens are suitable, in particular those having a SMS laminate structure, or SMMS, or SSMMS, structure. A core wrap layer may typically have a basis weight of from about 5 gsm to about 15 gsm. Suitable materials are for example disclosed in U.S. Pat. No. 7,744,576, US2011/0268932A1, US2011/0319848A1 and US2011/0250413A1. Nonwoven materials provided from synthetic fibers may be used, such as PE, PET and in particular PP. Higher basis weight material, in particular having a basis weight of at least 40 gsm, may also be used if the core wrap layer has other function, such as a masking layer for the bottom layer of the core wrap.

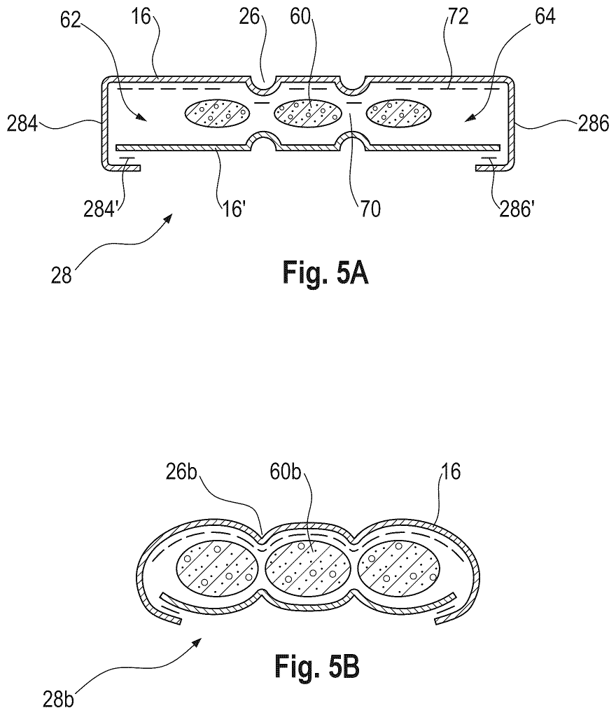

The core wrap may, as shown in the cross-sectional view of FIG. 5, comprise a first material forming the top layer 16 and a second material forming the bottom layer 16′ of the core. The top layer may advantageously be more hydrophilic than the bottom layer, for example the top layer may be treated with a wetting agent. Alternatively, it is also known in the art to make a core wrap out of a single substrate. When two discrete substrates are used, they may be formed with a C-wrap seal 284′, 286′ along each longitudinal edges 284, 286 of the absorbent core, e.g. as illustrated in FIG. 5. The longitudinal C-wrap seals 284′, 286′ may be formed by an adhesive, but other core wrap seals such as fusion bonded or other core wrap construction may be used in the present invention. The core wrap may optionally also have a front edge seal 280′ and a back edge seal 282′, which may be formed by adhesive or fusion bonds. The total length of the core wrap may be from about 5 to about 10 mm longer in the front and in the back than the absorbent layer, providing sufficient space for a sandwich front—and back core wrap end seals 284′, 286′. Examples of core wrap construction are further for example detailed in WO2014/093310. The core wrap is not considered as absorbent material for the purpose of calculating the percentage of SAP in the absorbent core.

The absorbent core may advantageously comprise one or more core channel(s) 26 within the absorbent layer 60, comprising a core wrap bond 70, as will be detailed further below.

### Absorbent Layer 60

The absorbent layer 60 is disposed between the core wrap top layer 16 and the core wrap bottom layer 16′. The absorbent layer may be formed by a single layer of absorbent material, or the absorbent layer may be comprised of two or more sub-layers which are combined within the core wrap.

The absorbent layer has a length L′ measured along the longitudinal axis 80′. The absorbent layer may be continuous, as represented, but it is not excluded that it may be segmented in sub-areas for example separated by transversally-orientated distribution channels to provide more flexibility in the longitudinal direction. The length L′ of the absorbent layer may typically be ranging from 50% to 90% of the length L of the absorbent article as a whole.

The absorbent layer of the absorbent core is typically substantially symmetrical relative to the longitudinal axis 80′ of the core. As exemplarily illustrated in FIG. 3, the absorbent layer 60 has a front edge 600, a back edge 602 and two longitudinal edges 604, 606 joining the front edge and back edge. The absorbent layer has a maximum width W1, which is typically the width of the front edge and/or the back edge, although other configurations are possible. For example while the front edge and the back edge may be generally straight and aligned with the transversal direction, it is not excluded that they may have other shape, such as being generally curved.

The absorbent layer 60 is not rectangular but instead comprises a narrow portion 612 at a position disposed between the front edge 600 and the back edge 602 of the absorbent layer 60. The narrow portion 612 consists of a longitudinally-extending portion of the absorbent layer having a reduced width relative to the rest of absorbent layer, in particular relative to the front portion 614 of the absorbent material layer (extending from the front edge of the narrow portion 612 to the front edge 600 of the absorbent layer) and the back portion 616 (extending from the back edge of the narrow portion 612 to the back edge 602 of the absorbent layer). The narrow portion 612 has a minimum width W2. The longitudinal edges 604, 606 of the absorbent layer may be substantially straight and parallel to the longitudinal direction in the front portion 614 and/or the back portion 616, outside of the narrow portion 612. The overall shape of the absorbent layer 60 may typically be a dog-bone or hour-glass shape, when viewed from the top of the core laid flat as illustrated in FIG. 3. The narrow portion 612 of the absorbent layer may have a length (measured along the longitudinal direction) ranging from 10% to 100% of the length L′ of the absorbent layer, in particular from 20% to 90% of the length L′, more particularly from 30% to 80% of the length L′.

The absorbent core comprises two absorbent material-free side areas 62, 64 disposed respectively transversally outwardly of the longitudinal edges 604, 606 of the absorbent layer in the narrow portion 612.

The narrow portion 612 of the absorbent layer 60 has a minimum width W2, as measured in the transversal direction at a longitudinal position intermediate between the front edge 600 and back edge 602. The absorbent layer's maximum width W1 is at least 10% larger than the absorbent layer's minimum width W2, as summarized by the formula below:

W1≥1.1×W2

Furthermore, it may be advantageous that W1 remain at most 200% larger than W2, as summarized by the formula below:

2.0×W2≥W1

The ratio W2/W1 may thus range from 1.1 to 2.0, in particular from 1.1 to 1.8, more particularly from 1.1 to 1.6.

The width of the absorbent layer 60 may be constant in the front portion and/or back portion of the absorbent layer, these portions being disposed longitudinally outside the narrow portion 612, but other configurations are possible. For example the width of the narrow portion may continuously expand towards the front and/or back edges of the core from the area having the minimum width W2. The narrow portion 612 may thus in some cases extend close to the front or the back edge of the absorbent layer. However the absorbent layer according to the invention is not rectangular.

The narrow portion 612 of the absorbent layer having the narrowest width W2 is advantageously disposed in the crotch region 37 of the absorbent article, which is central third of the article (as shown in FIG. 1). Advantageously, at least 50%, in particular at least 60% or 70%, of the length of the narrow portion 612 may be comprised in the crotch region 37 of the absorbent article. The area of the narrow portion 612 having the minimum width W2 is disposed in the crotch region 37 of the absorbent article.

The narrow portion 612 may be particularly flexible due to the narrow width of the absorbent material layer and the optional presence of core channel(s). This reduced width helps the absorbent core assuming a basin shape when the absorbent article is disposed on the wearer, with the front portion and back portion of the core respectively bending upwards towards the belly and buttocks of the wearer. This flexibility on the crotch portion of the core can help the barrier leg cuffs to maintain a contact with the wearer's thighs. The absorbent material-free side areas 62, 64 disposed outwardly of the longitudinal edges 284, 286 of the absorbent layer in the narrow portion 612 can also reach a higher position of the thighs of the wearer, thus increasing the effective cuff heights of the articles in which the absorbent core is integrated.

### Absorbent Material

The absorbent layer comprises an absorbent material that can absorb urine or other bodily exudates such as menstrual fluid. The absorbent material comprises superabsorbent polymer particles optionally mixed with cellulose fibers. The term “superabsorbent polymer” (herein abbreviated as “SAP”) refers to absorbent materials that can absorb at least 15 times their weight of an aqueous 0.9% saline solution as measured using the Centrifuge Retention Capacity (CRC) test (EDANA method WSP 241.2-05E). The SAP may in particular have a CRC value of from 20 g/g to 50 g/g, or from 25 g/g to 40 g/g. The absorbent material comprises SAP particles but other forms are also possible, such as absorbent foam or fibers. SAP are typically cross-linked acrylate polymeric material. Further detailed examples of absorbent material, in particular SAP, are disclosed in WO2014/093310 (Ehrnsperger). The absorbent material may also comprise or consist of SAP particles that require a time to reach an uptake of 20 g/g (T20) of less than 240 s as measured according to the K(t) test method described in WO2012/174026A1 (Ehrnsperger). The SAP particles used may have a permeability at equilibrium expressed as UPM (Urine Permeability Measurement) value of at least 10×10⁻⁷ (cm³·s)/g, in particular at least 15×10⁻⁷ (cm³·s)/g, or at least 20×10⁻⁷ (cm³·s)/g, or from 10 to 50×10⁻⁷ (cm³·s)/g, as measured by the test method indicated in WO2012/174026A1.

The absorbent material advantageously comprises a high proportion of superabsorbent polymer particles. The absorbent material may be a blend of cellulose fibers and superabsorbent polymer particles, typically with the percentage of SAP ranging from about 50% to about 75% by total weight of the absorbent material. The absorbent material may also be substantially free of cellulose fibers, comprising less than 15% by weight of cellulose fibers relative to the total weight of absorbent material, in particular less than 10%, or less than 5% and down to 0% by weight of cellulose fibers. The absorbent material may thus also be free of cellulose fibers, as is known in airfelt-free cores, where the absorbent material consists of, or consists essentially of SAP.

The absorbent core may be relatively thin, in particular thinner than conventional cores comprising cellulosic fibers. In particular, the caliper of the core (before use) as measured at the point corresponding to the crotch point C, or advantageously at any points of the surface of the core, may be less than about 5.0 mm, in particular from 0.2 mm to 4.0 mm, or from 0.5 mm to 4.0 mm, as measured according to the Thickness Measurement Method described further below.

The absorbent material may be substantially continuously distributed in the absorbent layer, as exemplary illustrated in FIG. 3. The basis weight of the superabsorbent polymers may also be distributed in the absorbent layer so that there is more SAP in the front half of the absorbent layer than in the back half of the absorbent layer.

The absorbent layer may be formed by any known processes that allow relatively precise deposition of absorbent material, in particular SAP polymers, advantageously at relatively high speed. For absorbent material comprising cellulose fibers mixed with the SAP polymers, the absorbent material is typically mixed and carried by an air flow within a mixing chamber into a moving mold comprising a 3D insert, the 3D insert being the inverse shape of the desired distribution pattern. Several molds are typically placed on a revolving circular drum to allow for continuous making of the absorbent core. The surface of the mold is in fluid communication with an under-pressure source in the deposition chamber except for the 3D insert (see e.g. EP3,342,386). A second substrate is then used to close the core wrap. The second substrate may be a second portion of the first substrate which is then folded over the deposited absorbent material to finish forming a core wrap, or it may be another substrate layer that is applied over the first substrate to form the absorbent core.

For absorbent material consisting essentially of superabsorbent polymer particles without cellulose fibers, a substantially continuous layer of absorbent material may for example be obtained by assembling two discontinuous absorbent sub-layers in the SAP printing process as disclosed in US2006/024433 (Blessing), US2008/0312617 and US2010/0051166A1 (both to Hundorf et al.). This technique uses a transfer device such as a printing roll to deposit SAP particles onto a substrate disposed on the grid of a support (e.g. a lay-on drum). The printing roll comprises a plurality of cavities arranged in cross-direction lines that can be filled with superabsorbent particles, the position, size and numbers of cavities can be adapted to deposit different quantities of particles according to the desired CD and MD profiles for the absorbent layer. The grid may include a plurality of cross bars extending substantially parallel to and spaced from one another so as to form ribs extending between the cross-bars. The SAP is deposited in the undulations of the substrate inside these ribs.

As known in the art indicated above (see e.g. EP2,949,299, Bianchi et al.), two such SAP printing roll/laying-on drum systems working in parallel can be used to deposit a SAP layer on each of the two substrates, the substrates being then assembled with the SAP layers in contact with each other thus forming a continuous layer of SAP between the core wrap's top layer and a bottom layer (formed by the two substrates). This SAP printing technology allows high-speed and precise deposition of SAP on a substrate in a desired pattern. The printing roll and the lay-on drum are adapted to provide the desired SAP application pattern, in particular shaping the absorbent layer. Optionally, raised elements on the transfer device may collaborate with corresponding mating strips on the support grid to provide areas free of deposited absorbent material within the absorbent layer. The top and bottom layers of the core wrap can be bonded together through some of these material-free areas to form the channels 26 discussed below. Thus, a SAP printing technique may be used to make absorbent cores according to the invention, for example as disclosed in US2006/024433 (Blessing), US2008/0312627 and US2010/051166A1 (both to Hundorf et al.) and US2014/027066A1 (Jackels) but of course it is not excluded that other manufacturing techniques may be used, or that products are hand-made for research purpose for example.

### Auxiliary Glue 72

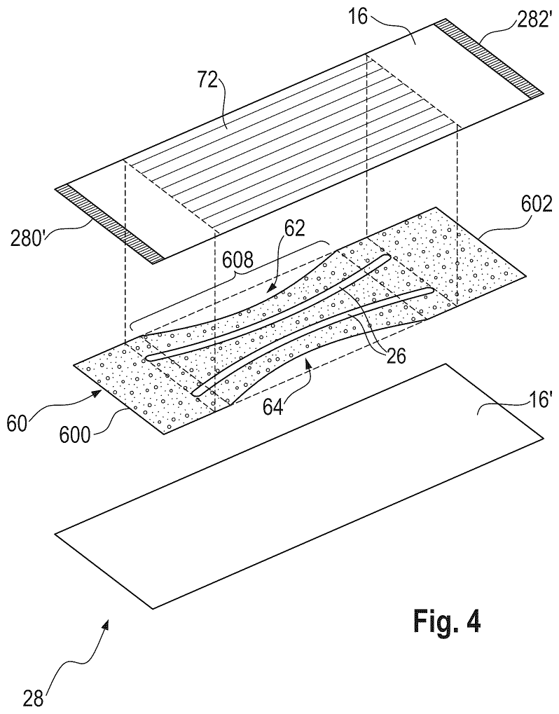

The absorbent core advantageously comprises one or more layers of glue to help immobilizing the absorbent material in position in the dry state. The absorbent core may comprise at least one auxiliary glue layer 72 applied on the inner surface of the top layer and/or the bottom layer of the core wrap as represented on FIGS. 4-5. The auxiliary glue 72 may at least partially immobilize the absorbent material on the substrate on which it is applied. The auxiliary glue may also be useful to improve the adhesion of a fibrous thermoplastic material used to immobilize the SAP particles to the substrate in airfelt-free cores.

The auxiliary glue 72 can be applied by any adhesive applicator known in the field, in particular bead, slot or spray nozzles. For example, the auxiliary glue can be applied using a slot coating process as a pattern comprising a plurality of spaced-apart slots which may each extend in the longitudinal direction. The slots may for example have a width of from 0.5 mm to 3 mm, typically 1 mm and/or have a lateral spacing there-between of from 0.5 mm to 4 mm, typically 1 mm. The auxiliary glue may be applied along the whole length of absorbent layer, or intermittently along a shorter length.

The auxiliary glue area may advantageously have a length which is at least 50% of the length L′ of the absorbent layer, in particular from 60% to 100% of the length L′ of the absorbent layer. As represented in FIG. 4, the auxiliary glue 72 is advantageously present in the narrow portion area 612 of the absorbent layer. The auxiliary glue 72 may also advantageously extend towards and including the front edge 600 of the absorbent layer 60 (not shown in FIG. 4), as typically more absorbent material is present towards the front of the core than towards the back.

The width of the auxiliary glue 72 may be substantially the absorbent layer′ maximum width W1 of the, in particular at least 90%, or at least 95%, or at least 100% of the width W1. This provides adhesion of the top layer and bottom layer of the core wrap also in the material-free side areas 62, 64 defined by the recesses 604, 606 of the longitudinal edges in the narrow portion 612.

The top layer 16 of core wrap may be adhered to the bottom layer 16′ of the core wrap by auxiliary glue 72 in the absorbent material-free side areas 62, 64. However this bond between the two layers may be preferably not as strong as the bond 70 in the core channel(s) 26. In this manner, while the auxiliary glue still has the effect of stabilizing the absorbent layer in the dry state, as the absorbent material swells, it may more easily expend sideways during use into the absorbent material-free areas 62, 64, (as illustrated in FIG. 5b),

The absorbent core may also comprise a fibrous thermoplastic adhesive material (not shown), also known as microfibrous glue, to help immobilizing the absorbent material within the core wrap, especially for airfelt-free cores. The fibrous thermoplastic adhesive material may be applied, typically by spraying, over an absorbent material that has been discontinuously deposited on a substrate during the core making process, thus forming land and junction areas as indicated above. The fibrous thermoplastic adhesive material contacts the absorbent material and the substrate layer in the absorbent material free junction areas. This imparts an essentially three-dimensional net-like structure to the fibrous layer of thermoplastic adhesive material, which in itself is essentially a two-dimensional structure of relatively small thickness, as compared to the dimension in length and width directions. Thereby, the fibrous thermoplastic adhesive material may provide a net-like cover over the absorbent material, and thereby immobilizes this absorbent material. A dual layer core can thus be constructed wherein the land areas of one layer correspond to the material-free junction areas of the other layer and vice versa, resulting in continuous dual absorbent layer.

The adhesive material may advantageously help providing a high immobilization of the absorbent material in dry and wet state. The absorbent core advantageously achieves an SAP loss of no more than about 70%, 60%, 50%, 40%, 26%, 20%, or 10% according to the Wet Immobilization Test described in US2010/051166A1.

### Core Channel 26

The absorbent layer 60 may comprise at least one longitudinally extending core channel 26. A core channel is a macroscopic area within the absorbent layer that is substantially free of absorbent material and is surrounded by absorbent material. By “substantially free”, it is meant that absorbent material is not deposited on purpose in these areas, but accidental contamination by some absorbent material such as SAP particles during the making process is not excluded. “Longitudinally extending” means that the core channel extends more in the longitudinal direction that in the transversal direction, preferably at least twice as much. The core channel(s) may have a length as measured in the longitudinal direction that is at least 10% of the length L′ of the absorbent layer, in particular from 20% to 90% of the length of the absorbent layer.

The top layer 16 of the core wrap is preferably bonded to the bottom layer 16′ of the core wrap through the absorbent material free area(s) forming the channel, as illustrated in FIG. 5. Typically, the core wrap bond 70 within the channel may be formed by any of an adhesive, a fusion bond, a mechanical bonding or a combination thereof. For example, the auxiliary glue 72 disposed on at least one of the top layer or the bottom layer of the core wrap may at least partially form the channel bonds 70. Applying pressure on the core wrap in the channels while the hotmelt adhesive is still molten further helps forming stronger bonds. When the absorbent layer 60 absorbs a fluid, e.g. urine, the absorbing absorbent material 60b surrounding the channel(s) can increase volume significantly so that three-dimensional depressions 26b are formed along the absorbent material-free area(s) 26.

The auxiliary glue 72 may at least partially form a core wrap bond 70 through the core channel(s) 26, especially in combination with pressure being applied in the core channel(s) or combined with another bonding means, for example ultrasonic bonding of the top layer and the bottom layer of the core wrap. Advantageously, the length of the auxiliary glue 72 is thus at least sufficient to cover at least the core channel(s) 26, if any are present in the absorbent core.

The core channels 26 can distribute the fluid along the length of the core in the absorbent layer. As illustrated in FIGS. 3-5, for example a pair of core channels 26 may be disposed symmetrically relative to the longitudinal axis 80′ in the absorbent layer 60. Exemplary disclosures of core channels in an airfelt-free core can be found in WO2012/170778 (Rosati et al.) and US2012/0312491 (Jackels). Channels may of course also be formed in absorbent cores comprising a mix of cellulose fibers and SAP particles.

The core channels 26 may be curved towards the longitudinal axis 80′ as illustrated in FIG. 3. The core channels may be curved along a smooth curve without inflexion points, as in a couple of inverted brackets: ) (. If curved core channels 26 are present, they may advantageously have substantially the same curvature as the longitudinal edges 284, 286 of the absorbent layer in the narrow portion 612 of the absorbent layer.

These core channel(s) may however embody any suitable shapes and any suitable number of channels may be provided. Many channel shapes have been suggested in the art. For example, at least a portion of the channel(s) may be aligned with the longitudinal axis 80′. In other instances, the absorbent core may be embossed to create the impression of channels.

The core channels 26 can increase the flexibility in the transversal direction of the absorbent core so that the absorbent core can more easily form a three-dimensional basin shape, when the article in which it is incorporated is placed between the thighs of a wearer. The width of the core channel(s) 26 may be substantially constant along the length of the core channel(s). The channels may for example have a width in the range of from 1 mm to 20 mm, in particular from 4 mm to 12 mm. The width may also vary along the length of the core channels. The channels may have a length, as measured in the longitudinal direction, which is at least 10% of the length L′ of the absorbent layer 60, typically from 15% to 80% of the length of the absorbent layer 60.

The three-dimensional depressions 26b in the absorbent core start forming when the absorbent material surrounding the core channels absorbs a liquid such as urine and starts swelling. As the core absorbs more liquid, the depressions within the absorbent core formed by channels will become deeper and more apparent to the eye and the touch. On the other hand, the top layer and the bottom layer of the core wrap may be completely unbonded, or at least not as strongly bonded, in the absorbent material-free side areas 62, 64. This allows the swollen absorbent material to extend sideways from the crotch portion 612 of the absorbent core. At some point, however the core wrap bond(s) may also be designed to open in a controlled manner when exposed to a large amount of fluid. The core wrap bonds 70 may thus remain substantially intact at least during a first phase as the absorbent material absorbs a moderate quantity of fluid and can expand sideways in the side areas 62, 64. In a second phase, the channel core wrap bonds 70 can start opening to provide more space for the absorbent material to swell while keeping most of the benefits of the channels such as increased flexibility of the core in transversal direction and fluid management. In a third phase, corresponding to a very high saturation of the absorbent core, a more substantial part of the channel bonds can open to provide even more space for the swelling absorbent material to expand. The strength of the core wrap bond within the channels can be controlled for example by varying the amount and nature of the adhesive used for the attaching the two sides of the core wrap.

### Liquid Management Layers 52, 54

Liquid management layers are disposed between the topsheet and the absorbent core and are designed to quickly acquire and/or distribute the fluid away from the topsheet and into the core. These liquid management layers are sometimes called “wicking layer”, “surge layer”, “acquisition layer”, “distribution layer” or “acquisition-distribution layer”. Liquid management layers do not comprise SAP, as this may slow the acquisition and distribution of the fluid. The prior art discloses many type of liquid management layer, see for example WO2000/59426 (Daley), WO95/10996 (Richards), U.S. Pat. No. 5,700,254 (McDowall), WO02/067809 (Graef).

The absorbent article of the invention comprises at least one fibrous liquid management layer between the topsheet 24 and the absorbent core 28, referred herein as distribution layer 54. Optionally, the absorbent article may comprise an additional liquid management layer, referred herein as acquisition layer 52, disposed between the topsheet and the distribution layer. The distribution layer may be profiled in the longitudinal direction and/or have a generally rectangular shaped with a rounded end (“bullet” shaped) towards the back of the diaper. Such dual layer liquid management layers are for example disclosed in further details in WO2014/093323 (Bianchi et al.).

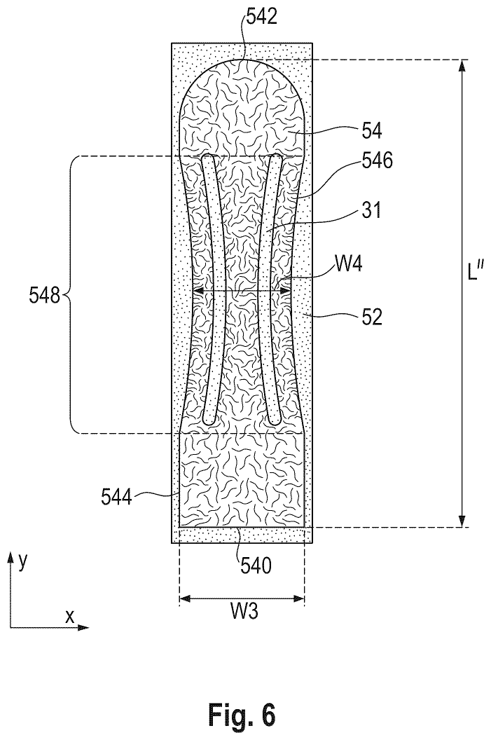

As illustrated in FIG. 6, the fibrous distribution layer 54 has a front edge 540, a back edge 542 and two longitudinal edges 544, 546. The distribution layer 54 has a maximum width W3 (as measured in the transversal direction x). The maximum width W3 may typically be measured towards the front of the distribution layer. The distribution layer 54 has a length L″ (as measured in the longitudinal direction y along its longitudinal axis). According to the invention, the distribution layer 54 is not rectangular but comprises a narrow portion 548 between the front edge 540 and the back edge 542 of the distribution layer, the narrow portion 548 having a minimum width W4. The distribution layer's narrow portion 548 may have a length (measured along the longitudinal direction) ranging from 10% to 100% of the length L″ of the distribution layer, in particular from 20% to 90% of the length L″, more particularly from 30% to 80% of the length L″.

According to the invention, W3 is at least 10% larger than W4, as summarized by the formula:

W3≥1.1×W4.

Advantageously, the maximum width W3 is at most 60% larger than minimum width W4, as summarized by the formula:

1.6×W4≥W3

The ratio W4/W3 may thus range from 1.1 to 1.6, in particular from 1.1 to 1.5, more particularly from 1.1 to 1.4.

The narrow portion 548 of the distribution layer having the narrowest width W4 is advantageously disposed in the crotch region 37 of the absorbent article, which is central third of the article (as shown in FIG. 1). Advantageously, at least 50%, in particular at least 60% or 70%, of the length of the narrow portion 548 may be comprised in the crotch region 37 of the absorbent article. The length of the narrow portion 548 may for many applications ranges between 100 mm and 260 mm.

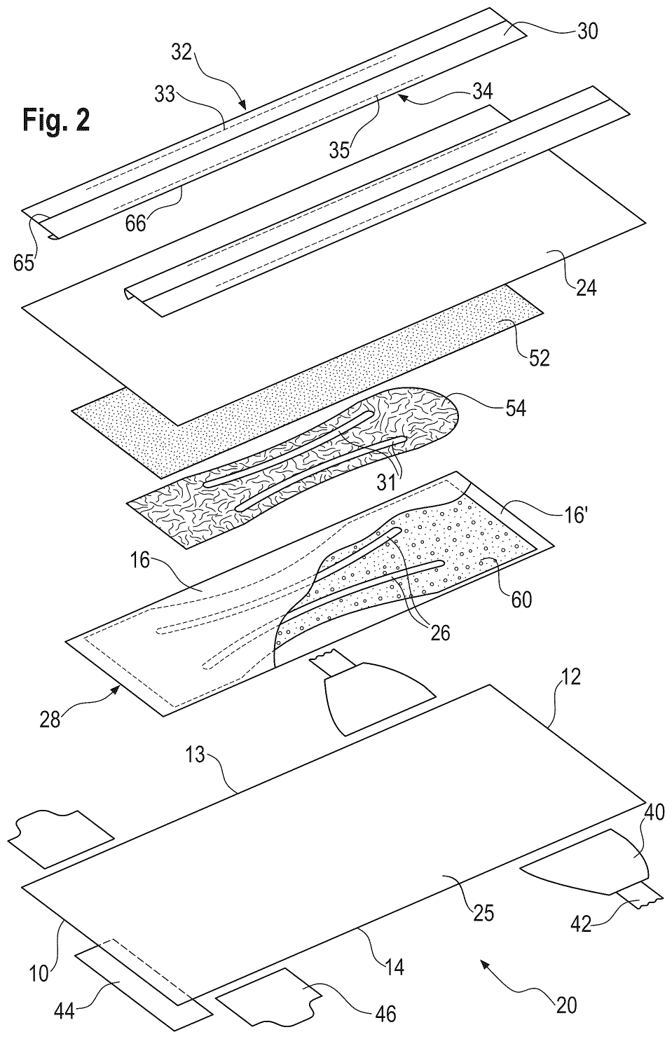

The distribution layer 54 may be provided with one or more distribution channels 31. The distribution channels 31 may be at least partially vertically superposed with the channels 26 of the absorbent core, when these are present, as illustrated in FIGS. 1-2. Thus, the distribution channel 31 can transfer a fluid directly to the channels 26 disposed underneath, providing a better fluid transfer away from the topsheet into the rest of the absorbent core. By “vertically superposed”, it is meant that the position and shape of the distribution channels 31 of the distribution layer vertically correspond to the underlying channels 26 of the absorbent core. The distribution channel(s) can also increase the flexibility of the absorbent article as a whole, for example helping the article to readily assume a basin-like shape formed by the underlying absorbent core when the article is put on and worn by the wearer.

The distribution channels 31 of the liquid management layer may be superposed with the channels 26 of the absorbent core over the whole length of distribution channels, but typically they may be shorter that the channel(s) of the absorbent core. The distribution layer's distribution channels may overlap over at least 50%, 60%, 70% or more of the overall length of the absorbent core's channel(s). In the remaining areas where there is no overlap, the distribution channels 31 may be longer that the core channel(s), or may be shorter and thus not extend to the same length as the absorbent core channel(s) 26.

The distribution channels 31 may be formed by areas substantially free of the fibrous distribution material, e.g. substantially free of unbound or loosely bound hydrophilic fibers such as cross-linked cellulosic fibers.

The longitudinal edges of the absorbent layer and/or the distribution layer are advantageously inwardly curved in the narrow portions of the respective layer. In particular, the curvature of the distribution layer and the absorbent layer may be substantially similar in the respective narrow portion. Additionally, if the distribution channels 31 are present, they may be curved and have substantially the same curvature as the longitudinal edges of the distribution layer in the narrow portion 548 of the distribution layer.

The function of a distribution layer is to spread the insulting fluid liquid over a larger surface within the article so that the absorbent capacity of the core can be more efficiently used. Typically, distribution layers can be made of a material comprising synthetic or cellulosic fibers and having a relatively low density. The distribution layer material may be a fibrous nonwoven or a fibrous layer comprising unbound or loosely bound hydrophilic fibers, in particular a layer of cross-linked cellulosic fibers. The density of the distribution layer may vary depending on the compression of the article, but may typically range from 0.03 g/cm³ to 0.25 g/cm³, in particular from 0.05 g/cm³ to 0.15 g/cm³ measured at 0.26 psi (2.07 kPa). The distribution layer may also be a material having a water retention value of from 25 to 60, preferably from 26 to 45, measured as indicated in the procedure disclosed in U.S. Pat. No. 5,137,537.

Such a distribution layer may for example comprise at least 50% by weight, optionally consisting of 100%, of cross-linked cellulosic fibers. The cross-linked cellulosic fibers may be crimped, twisted, or curled, or a combination thereof including crimped, twisted, and curled. This type of material has been used in the past in disposable diapers as part of an acquisition system, for example US2008/0312622 A1 (Hundorf), however not in the manner of the invention. The cross-linked cellulosic fibers provide higher resilience and therefore higher resistance against the compression in the product packaging or in use conditions, e.g. under baby weight. This provides the layer with a higher void volume, permeability and liquid absorption, and hence reduced leakage and improved dryness. The liquid management layer 54 may also be typically profiled so that more material is present at the front and middle part of the article relative to the back of the article. The distribution layer may typically have an average basis weight of from 26 g/m² to 400 g/m², in particular from 100 g/m² to 260 g/m², with the basis weight varying along the length of the article so that more material is present at the front and middle of the layer than at the back. The liquid management layer may thus be profiled and/or shaped rounded towards the back of the article, as exemplarily disclosed in WO2014/093323 (Bianchi).

A fibrous distribution layer 54 may for example be made on-line by depositing cross-linked cellulosic fibers on a forming surface. The forming surface may comprise ridges corresponding to the areas where no fibrous material is desired to form distribution channels 31, which are areas free of distribution material. Deposition chambers are known wherein a carrier sheet is provided on a forming surface having a series of holes connected to a vacuum, so that the vacuum pulls the fibers in the desired emplacements to form a desired deposited layer. The forming surface of these deposition chambers can be modified to provide a layer of fibrous material having a central portion and side portions separated by distribution channels. The fibrous layer is typically formed or transferred on a carrier sheet that should thus have at least the same dimension as a fibrous liquid management layer. The carrier sheet may be the topsheet, another liquid management layer such as a nonwoven acquisition layer, or any other layer of the article, for example the core wrap. An adhesive (not represented), in particular a hotmelt adhesive, may be typically applied on a substrate on which the loose fibers of the distribution layer are deposited, in particular such adhesive may be applied on the acquisition layer to at least partially immobilize the fibers of the distribution layer.

The acquisition layer 52 may be made of a nonwoven web having hydrophilic properties. The nonwoven web may be for example provided as a continuous roll of material that is cut according to the desired length and pattern as it is unwound in a converting line. Such an acquisition layer is typically placed directly under the topsheet. Suitable nonwovens are for example through-air bonded (“TAB”) carded nonwovens, resin-bonded (“RB”) carded nonwovens, spunbond or spunlace (hydroentangled) nonwovens. TAB carded nonwovens may for example be made from soft PE/PP bicomponent staple fibers. The air through bonding process locks in loft and compressibility. Resin-bonded carded nonwovens may be made from multi-denier polyester staple fibers (for example: 50/50 or 40/60 mix of 6 denier and 9 denier fibers). Its resilient and open structures are designed to provide excellent fluid acquisition properties. Such acquisition layers are available directly from suppliers, e.g. Fitesa of Simpsonville, South Carolina, USA or TWE Group GmbH, of Emsdetten, Germany. The nonwoven layer may be stabilized by a latex binder for example a styrene-butadiene latex binder (SB latex). Processes for obtaining such latexes are known, for example from EP149,880 (Kwok), US2002/028858 and US2003/0105190 (Diehl). The binder may typically be present in an acquisition layer in excess of about 12%, about 14% or about 16% by weight of the layer. A SB latex is for example commercially available under the trade name GENFLO™ 3160 (OMNOVA Solutions Inc.; Akron, Ohio). Latex bonded acquisition layers are for example further disclosed in US2005/033252A1, US2005/033253A1 or US2005/043694A1 (Schneider). The basis weight of typical acquisition layers 5 ranges from 10 gsm to 200 gsm, in particular 20 gsm to 140 gsm, or 40 gsm to 120 gsm, for example 80 gsm.

Since the function of a liquid management layer is to help transfer the insulting fluid to the absorbent material, it is generally not desirable for the distribution layer 54 to extend beyond the transversal and longitudinal edges of the absorbent layer of the absorbent core.

An acquisition layer 52 may be used in the diaper of the invention having a generally rectangular shape and having a width which is at least equal to the maximum width W3 of the distribution layer 54.

Advantageously, the acquisition layer 52 should not hinder or provide a significant resistance to the folding or flexing of the absorbent article. The acquisition layer may thus be sufficiently conformable and flexible for this purpose. For example the acquisition layer 52 may be a nonwoven having a basis weight of less than 200 g/m², typically from 26 g/m² to 120 g/m², in particular from 26 g/m² to 80 g/m² for example a spunlace nonwoven layer, or an air-through bonded or latex bonded carded layer.

The invention is however not restricted to the above embodiments having two liquid management layers, as a fibrous distribution layer 54 maybe also used without an acquisition layer 52. This is especially the case if the fibrous distribution layer is a nonwoven having sufficient integrity so that it does not require a substrate for deposition. Suitable nonwovens having distribution properties are for example spunlace. The term “spunlace” means a nonwoven wherein the cohesion and the interlacing of the fibers with one another is obtained by means of a plurality of jets of water under pressure passing through a moving fleece or cloth and, like needles, causing the fibers to intermingle with one another. These spunlace nonwovens are essentially defined by the fact that their consolidation results from hydraulic interlacing. “Spunlace”, as used herein, also relates to a nonwoven formed of two or more webs (stratum), which are combined with each other by hydraulic interlacing. The two webs, prior to being combined into one nonwoven by hydraulic interlacing, may have underdone bonding processes, such as heat and/or pressure bonding by using e.g. a patterned calendar roll and an anvil roll to impart a bonding pattern. However, the two webs are combined with each other solely by hydraulic interlacing.

The spunlace may comprise a plurality of absorbent fibers, a plurality of stiffening fibers and a plurality of resilient fibers, in particular, by weight of the nonwoven:
from about 20 percent to about 75 percent of absorbent fibers;
from about 1 percent to about 50 percent of stiffening fibers;
from about 10 percent to about 50 percent of resilient fibers.

Examples of suitable spunlace are disclosed in WO2020/205485 and WO2021/119653A1.

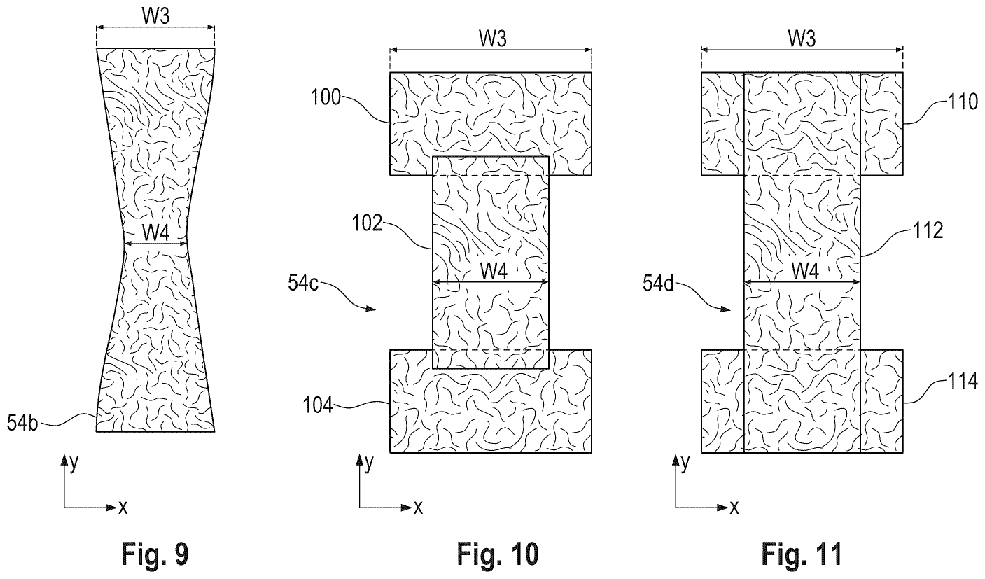

The spunlace, or any other fibrous distribution material used, may be a rectangular piece of material that is trimmed along it longitudinal edges to form a narrow portion having a minimum width W4. Such first trimmed distribution layer 54b is exemplarily presented in FIG. 9. Alternatively, it is also possible to combine rectangularly shaped piece of distribution material (spunlace or otherwise) to form a non-rectangular distribution layer. FIG. 10 illustrates such a construction 54c comprising a front end segment 104 and a back end segment 100 consisting of a fibrous distribution material, wherein both segments are oriented in the transversal direction and at least one of these segments having a maximum width W3, and the transversally oriented segments are joined by a narrower and longitudinally-oriented middle segment 102 of a fibrous distribution material having a minimum width W4. FIG. 11 shows another alternative multi-piece construction 54d, wherein the central segment 112 is however as long as the fibrous distribution layer 54d as a whole and entirely overlaps lengthwise the front and back end segments 110, 114, thus providing additional distribution capacity at the front and back segments 110,114. Of course an intermediate construction is possible, wherein the middle segment only partially overlapping the front and/or back segment.

### Absorbent Sideline Areas 62a, 64a

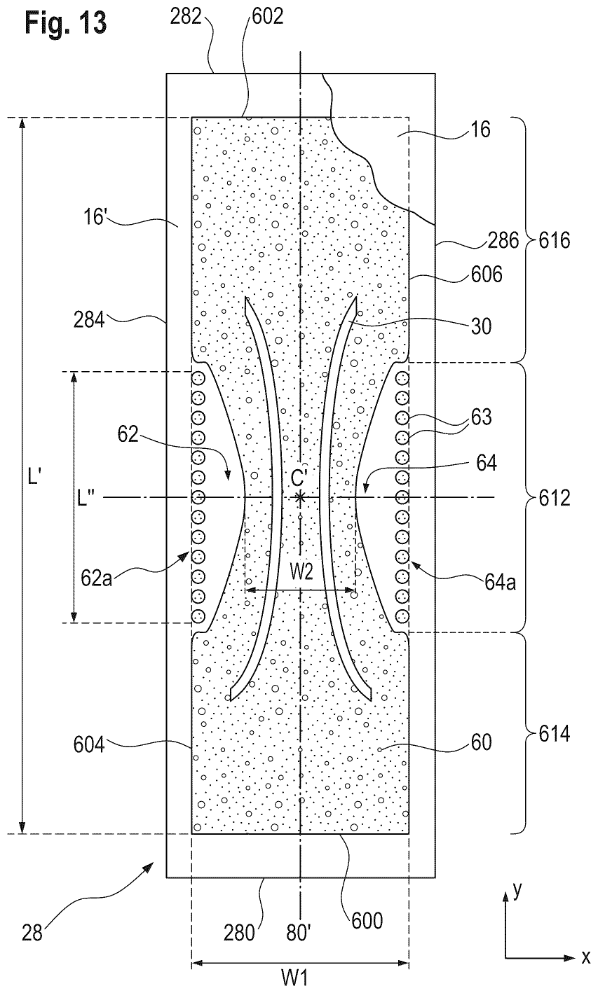

The side areas 62, 64 in the crotch portion 612 of the absorbent material area may be free of absorbent material, as illustrated in FIG. 3. Alternatively, the side areas 62, 64 may also comprise a first absorbent sideline area 62a and a second absorbent sideline area 64a, disposed respectively in the first side area 62 and second side area 64, on either side of the longitudinal axis 80′, as illustrated in FIGS. 12-13. The absorbent sideline areas 62a, 64a are longitudinally-extending and are disposed along the outward edges of the side areas 62, 62. The sidelines areas 62a, 64a may be generally longitudinally aligned with the longitudinally-extending side edges 604, 606 of the absorbent layer outside the central portion. The sideline areas 62a, 64a may be in particular aligned with the longitudinal direction y as illustrated in FIGS. 12-13.

The absorbent sideline area 62a, 64a may each be comprised by a plurality of absorbent material islands 63, each island being separated from the adjacent islands by a gap which is free of absorbent material (as seen from the top, when the absorbent core is dry and laid flat, as illustrated in FIG. 12). The plurality of islands 63 forming each sideline area may be generally longitudinally-aligned, in particular they may be aligned parallel to the longitudinal direction y. The islands 63 may also be aligned in the continuity of the longitudinally-extending side edges 604, 606 of the absorbent layer 60 outside the narrow portion 612, so that the absorbent material area as a whole has a generally rectangular perimeter when considering the absorbent material sideline areas 62a and 64a as a continuation of the longitudinal side edge 604, 606 of the absorbent layer 60 outside the narrow portion 612.

The dimensions of the islands and the gap between adjacent islands may vary or be the same for each island and gap. The average diameter of the islands may typically range from 2 mm to 10 mm, for example about 5 mm. The average gap between adjacent islands may be typically have about the same dimension as the island's diameter, typically ranging from 2 mm to 10 mm, for example or about 5 mm. The island may be substantially circular, but other shapes are possible. For non-circular island, the diameter is measured as the longest distance measurable going through the center of each island. It is understood that even modern manufacturing requirement do not allow to deposit perfectly circular islands of absorbent material at the high speed required. The substantially circular islands may thus also be described as blots or dots.

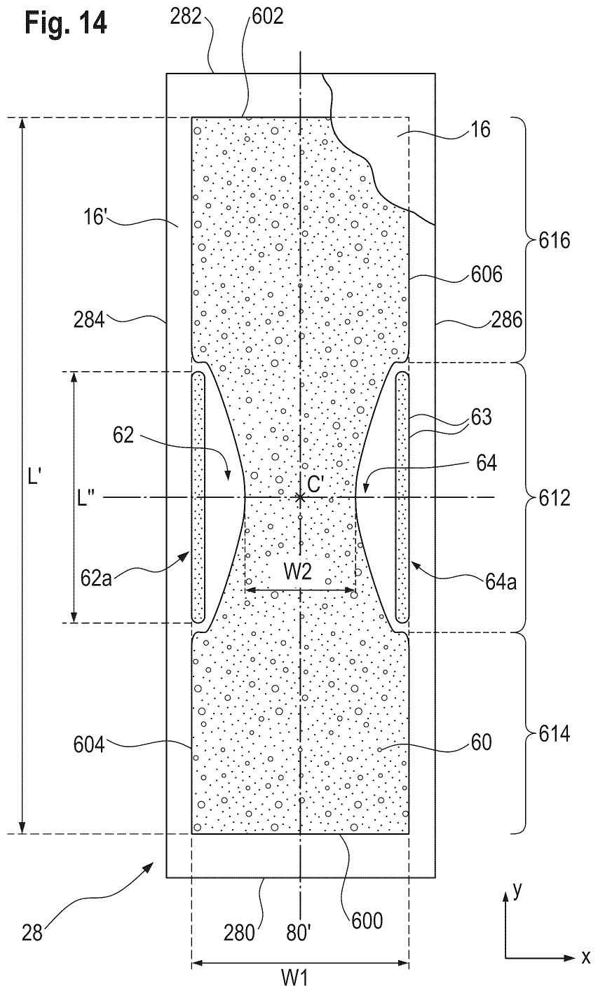

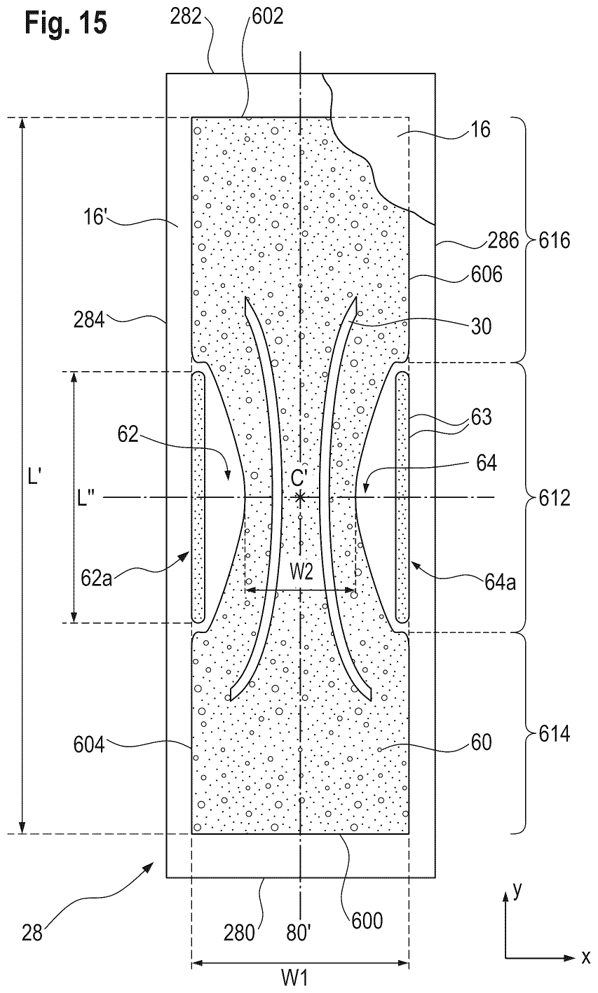

Alternatively, each of the absorbent sideline areas may each be formed as a continuous area 62b, 64b unconnected to the central layer 60, as illustrated in FIGS. 14-15. Further alternatively, the continuous sideline areas may even be connected to at least one or more of its extremities to the absent layer 60.

The length L″ of each absorbent sideline area 62a, 64a is measured in the longitudinal direction y. When the absorbent sideline areas are formed by several islands of absorbent material, as illustrated in FIG. 12, the length L″ from the first island to the last island. The islands may be substantially aligned with the longitudinal direction y, for example perfectly aligned as illustrated in FIG. 12, or aligned at an angle (e.g. up to 40 degree relative to the longitudinal direction), or distributed alternatively on either side of a line parallel to the longitudinal direction y. The length L″ may be ranging from about 25% to about 70%, or from 30% to 60%, of the length L′ of the absorbent material layer 60.

The sidelines areas are longitudinally-extending, so that their length is a multiple of their width. Typically the width of the sideline areas may by ranging from 2 mm to 2 cm, in particular from 3 mm to 10 mm. The length to width ratio of the sideline areas may typically range from 2 to 20, in particular from 4 to 14.

Providing absorbent sideline areas has several benefits. Firstly, the absorbent sideline areas 62a, 64a provide absorbent capacity within the otherwise absorbent material-free side areas 62, 64 in the narrow portion 612.

Secondly, the sideline areas reduce the risk of side leakage due to an acquisition layer 52 or a distribution layer 54 extending transversally outwardly of the core central portion 612. The acquisition layer 52 and/or distribution layer 54 may thus have a maximum width W3 which may extend to substantially the maximum width W1 of the absorbent material area, to reduce risk of side leakage at the narrow portion 612. The sideline areas can act as a barrier for a fluid running sideways.

Thirdly, when the absorbent sideline areas are present and comprise islands 63 separated by gaps free of absorbent material, the distance between adjacent islands may be reduced (up to contact between the islands), thus providing tighter sidewalls of protection against side leakages. The gaps between the absorbent material islands 63 are flexible as the core wrap material present therein can easily deform. When the absorbent core assumes a basin shape in use, the absorbent material islands can come closer to each other and the gaps between adjacent islands diminish. In some cases, the islands may contact each other when the article is worn by the wearer. This increases the fluid barrier properties of the sidelines when the article in use, especially when the absorbent material in the islands 63 starts absorbing urine and expand, further closing the gaps between the islands of absorbent material 63.

The longitudinal side edges 604, 606 of the absorbent material area 60 and/or the sideline areas 62a, 64a may extend to the longitudinal side edges 284, 286 of the core wrap. The front edge 600 and the back edge 602 of the absorbent material area 60 may or may not be spaced from the front edge 280 and back edge 282 of the core wrap respectively, as illustrated in FIG. 3, so that the core wrap may or may not comprise a front end seal and a back end seal in the terminal absorbent material-free areas thus formed at the front and back edge of the core. The top layer and bottom layer of the core wrap may be bonded in these terminal areas to prevent absorbent material from escaping the core wrap from its front edge or back edge. The front edge 600 of the absorbent material area 60 may be coterminous or adjacent or separated from the front edge 280 of the absorbent core. The back edge 602 of the absorbent material area 60 may be coterminous or adjacent or separated from the back edge 282 of the absorbent core. The core wrap may optionally be sealed along any of its sides, or its whole periphery. The core wrap may in particular comprise side seals 284′, 286′ along the longitudinally-extending side edges of the core, which may be sandwich seals or C-wrap seals as illustrated in FIG. 5. However, it is also possible that there is no core wrap seal on one or more sides of the absorbent core, for example along the front edge and back edge of the core.

### Topsheet 24

Any topsheet known in the art for absorbent articles may be used for the articles of the invention. The topsheet is preferably compliant, soft-feeling, and non-irritating to the wearer's skin. Further, at least a portion of the topsheet is liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet includes fibers, the fibers may be spunbond, carded, wet-laid, meltblown, hydroentangled, or otherwise processed as is known in the art, in particular spunbond PP nonwoven. A suitable topsheet comprising a web of staple-length polypropylene fibers is manufactured by Veratec, Inc., a Division of International Paper Company, of Walpole, MA under the designation P-8. Typical diaper topsheets have a basis weight of from about 10 to about 28 gsm, in particular between from about 12 to about 18 gsm but other basis weights are possible.

Suitable formed film topsheets are also described in U.S. Pat. Nos. 3,929,135, 4,324,246, 4,342,314, 4,463,045, and 5,006,394. Other suitable topsheets may be made in accordance with U.S. Pat. Nos. 4,609,518 and 4,629,643. Such formed films are available from The Procter & Gamble Company of Cincinnati, Ohio as “DRI-WEAVE” and from Tredegar Corporation, based in Richmond, VA, as “CLIFF-T”.

The topsheet may also be treated with a wetting agent to increase its more hydrophilicity. The wetting agent may be a surfactant, as is known in the art. Other possible treatments are for example special coating by nanoparticles, as for example described in U.S. Pat. Nos. 6,645,569, 6,863,933, US2003/148684 and US2005/008839, (Cramer et al.) and U.S. Pat. No. 7,112,621 (Rohrbaugh et al). Any portion of the topsheet may also coated with a lotion as is known in the art. Examples of suitable lotions include those described in U.S. Pat. Nos. 5,607,760, 5,609,587, 5,643,588, 5,968,025 and 6,716,441. The topsheet may also include or be treated with antibacterial agents, some examples of which are disclosed in WO 95/24173. Further, the topsheet, the backsheet or any portion of the topsheet or backsheet may be embossed and/or matte finished to provide a more cloth like appearance.

The topsheet 24 may comprise one or more apertures to ease penetration of exudates therethrough, such as urine and/or feces (solid, semi-solid, or liquid). The size of at least the primary aperture is important in achieving the desired waste encapsulation performance. If the primary aperture is too small, the waste may not pass through the aperture, either due to poor alignment of the waste source and the aperture location or due to fecal masses having a diameter greater than the aperture. If the aperture is too large, the area of skin that may be contaminated by “rewet” from the article is increased. Typically, the total area of the apertures at the surface of a diaper may have an area of between about 10 cm² and about 50 cm², in particular between about 15 cm² and 35 cm². Examples of apertured topsheet are disclosed in U.S. Pat. No. 6,632,504. WO 2011/163582 also discloses suitable colored topsheet having a basis weight of from 12 gsm to 18 gsm and comprising a plurality of bonded points. Each of the bonded points has a surface area of from 2 mm² to 5 mm² and the cumulated surface area of the plurality of bonded points is from 10% to 25% of the total surface area of the topsheet.

Although not shown in the drawings, it is possible to bond the topsheet directly or indirectly to the absorbent core through the distribution channels. The topsheet may be bonded by any known bonding means, typically adhesive bonding, pressure bonding or heat bonding, or a combination of these to the underlying layer.

### Backsheet 25

The backsheet 25 may also be made according to any backsheet known in the art for absorbent articles. The backsheet 25 is typically impermeable to liquids (e.g. urine) so that it keeps the garment-facing side of the article dry. The backsheet may for example be or comprise a thin plastic film such as a thermoplastic film having a thickness of less than about 0.10 mm. Exemplary backsheet films include those manufactured by Tredegar Corporation, based in Richmond, VA, and sold under the trade name CPC2 film. Other suitable backsheet materials may include breathable materials which permit vapors to escape from the article while still preventing exudates from passing through the backsheet. A low basis weight nonwoven cover may be attached to the external surface of the film to provide a softer touch.

### Other Components of the Article

The absorbent articles of the invention can comprise any typical components known for the intended purpose of the article. FIGS. 1-2 show other some typical taped diaper components such as a fastening system comprising a pair of back ears 40 extending transversally outwardly adjacent the back edge 12 of the article and each having a fastening tab 42 attached thereto that can fasten to a landing zone 44 placed adjacent the external side of the front edge 10 of the article. These fastening features are typically absent from pant-type articles which have a pre-formed side seam, nevertheless the invention may of course also be used in such pant-types articles. The absorbent article may also comprise other typical components, which are not represented in the Figures, such as a back elastic waist feature, a front elastic waist feature, a transverse barrier element across the topsheet, a wetness indicator between the core and the backsheet that changes appearance when contacted with urine, a lotion application on the topsheet, etc. These components are well-known in the art and will not be further discussed herein. Reference is made to WO2014/093310 where several examples of these components are disclosed in more details.

The absorbent article may however in particular a pair of inner leg cuffs 34 and outer barrier leg cuffs 32, referred together as barrier leg cuffs. The barrier leg cuffs can provide improved containment of liquids and other body exudates approximately at the junction of the torso and legs of the wearer. Typically, the inner cuffs are at least partially formed from a separate material joined to the rest of the article, in particular to the topsheet, but it is not excluded that the inner cuffs can be integral with (i.e. formed from) the topsheet or the backsheet, or any other layer, for example the bottom layer of the core wrap. Typically the material of the barrier leg cuffs may extend through the whole length of the article but is further bonded to the topsheet towards the front edge and back edge of the article so that in these sections the barrier leg cuff material remains flush with the topsheet (tack bonds not shown in FIG. 1 for readability).

Each of the inner leg cuffs 34 comprises a proximal edge 65 attached to the chassis of the article and a distal edge 66 that can be raised away from the topsheet. The inner leg cuffs 34 can thus protrude away from the topsheet at least in the crotch region 37 of the article. The proximal edge 65 may be attached to the topsheet or another layer of the chassis with an intermittent or continuous fusion bond and/or a glue bond. The distal edge 66 of the inner cuffs is elasticized and is designed to fit at the junction of the thighs with the torso of the wearer. Each inner leg cuff 34 typically comprises one, two or more elastic strings 35 close to this free-standing distal edge 66. The elastic threads ensure that the inner cuff distal edge is under tension and remain upwards and form a good seal with the thighs of the wearer.

In addition to the inner leg cuffs 34, the article typically comprises outer cuffs 32, which are integrated with the chassis of the absorbent article. The outer cuffs may be at least partially enclosed between the topsheet and the backsheet, or as illustrated may be formed by the barrier leg cuff material 30 on one side and the backsheet 25 on the other side. The outer leg cuffs 32 are disposed transversally outwardly relative to the proximal edge 65 of the inner cuffs 34. The outer cuffs 32 provide a better seal around the thighs of the wearer. Usually each outer leg cuff 32 comprises one or more elastic string or elastic element(s) 33 embedded within the chassis of the diaper, for example between the topsheet and backsheet in the area of the leg openings, or between a barrier leg cuff material 30 and the backsheet 25. These elastic elements 33, independently or in combination with the elastics 35 of the inner cuffs, help shaping the absorbent article into a basin when the article is put in place on the wearer.

Various cuff constructions have been disclosed for in the art and may be used in the present invention. U.S. Pat. No. 3,860,003 describes a disposable diaper which provides a contractible leg opening having a side flap and one or more elastic members to provide gasketing cuffs. U.S. Pat. Nos. 4,808,178 and 4,909,803 (Aziz) describe disposable diapers having “stand-up” elasticized flaps (inner leg cuffs) which improve the containment of the leg regions. U.S. Pat. No. 4,695,278 (Lawson) and U.S. Pat. No. 4,795,454 (Dragoo) describe disposable diapers having dual cuffs, including outer leg cuffs and inner leg cuffs. More recently, WO2005/105010 (Ashton) discloses a dual cuff system made of a continuous cuff material.

The combined elastic forces provided by the different elasticized components of the article may thus bring or facilitate bringing the article into a basin shape when the article is placed on a wearer. Although not represented, the article of the invention may further comprise other longitudinally-extending elasticized elements as known in the prior art.

More generally, adjacent layers within the article will be joined together using conventional bonding method such as adhesive coating via slot coating, spiral gluing, or spraying on the whole or part of the surface of the layer, or thermo-bonding, or pressure bonding or combinations thereof. Most of the bonding between components is for clarity and readability not represented in the Figure. Bonding between the layers of the article should be considered to be present unless specifically excluded. Adhesives may be typically used to improve the adhesion of the different layers. For example, the backsheet and the core wrap may be glued using a core-to-backsheet gluing pattern as disclosed in WO2012/170341A1 (Hippe), or a full coverage pattern using several spiral glue applicators. If for example the backsheet is attached by gluing or otherwise to the areas of the core wrap corresponding to the distribution channels (not shown), the distribution channels may become more visible to the user from the garment-facing side of the article. Any typical hotmelt adhesives may be used. It is also possible to use a printed adhesive layer, for example between the topsheet and absorbent core or liquid management layer, which may be optionally visible through the topsheet, as exemplary disclosed in WO2014/078247.

Typically, adjacent layers and components of the absorbent article will be joined together using conventional bonding method such as adhesive coating (via slot coating, spiral gluing, or spraying) on the whole or part of the surface of the layer, or thermo-bonding, or pressure bonding or combinations thereof. Most of the bonding between components is for clarity and readability not represented in the Figure. Bonding between the layers of the article should be considered to be present unless specifically excluded. Adhesives may be typically used to improve the adhesion of the different layers, for example between the backsheet and the core wrap as disclosed in WO2012/170341A1. The adhesives used may be any standard hotmelt glue as known in the art. FIG. 5 shows in dotted lines the presence of the auxiliary adhesive 72 which was disposed on the core wrap top layer's inner surface and bonds the absorbent material and to the core wrap bottom layer directly in contact, as well as an adhesive providing for the longitudinally extending absorbent core side seals 284′, 286′.

### EXAMPLES

The present invention is applicable to a wide range of absorbent cores and absorbent articles. The dimensions and materials used may vary depending on the intended applications as is known in the art. For example, absorbent cores for larger size diapers will typically be longer and larger and comprise more absorbent material than absorbent cores for smaller size diapers.

### Example 1

The following illustrates dimensions and materials for an exemplary core as represented in FIGS. 1-6 and comprising a pair of core channels and a pair of distribution channels. The following are exemplary dimension values for an absorbent core suitable for a “size 4” (“Midi”) diaper, which can be recommended for baby weighing between 7 kg and 18 kg. All widths are measured parallel to the transversal direction (x) and all lengths are measured parallel to the longitudinal direction (y).
W1=110 mm
W2=90 mm
W3=80 mm
W4=64 mm

| | |
|---|---|
| L (length of the article) | 478 mm |
| L′ (length of the absorbent layer) | 360 mm |
| L612 (length of the core narrow portion) | 182 mm |
| Length of channel 26 (in y direction) | 227 mm |
| Width of channel 26 | 8 mm |
| L″ (length of the distribution layer) | 298 mm |
| L548 (length of the distribution layer narrow portion 548) | 176 mm |
| L31 (length of the distribution channels in y direction) | 173 mm |

In this example, the core wrap comprises a top layer C-wrapped around a bottom layer with adhesive longitudinal seals (as in FIG. 5). The top layer has a width of 165 mm, the bottom layer has a width of 126 mm. The final core bag's width is approximately 120 mm. The top layer is a 8 gsm water permeable propylene spunbonded nonwoven (from Fitesa Germany) and the bottom layer an hydrophobic, 10 gsm nonwoven (from Fibertex). The core wrap is sealed by fusion bond or gluing at its front edge and its back edge. The absorbent material consists entirely of SAP particles, such as L900, ex Nippon Shokubai Group, which is deposited for one half on the top layer and the other half on the bottom layer of the core wrap and immobilized by a microfibrous glue (SAP printing method as described above). An auxiliary slot glue layer is disposed on the inner side of the top core wrap layer with an overall width of 81 mm (slots 1 mm with 1 mm gap in-between). The total amount of SAP is 12.7 g. The basis weight of the SAP in the absorbent layer is advantageously longitudinally profiled so that more absorbency is available towards the middle and the front of the core. The total length of the core wrap is about 10 mm longer in the front and in the back than the length of the absorbent material layer to provide for SAP free sandwich front and back core wrap end seals.

The diaper incorporating the absorbent core may comprise a dual acquisition-distribution system comprising a latex bonded, nonwoven acquisition layer 52 and a cross-linked cellulose fibers distribution layer 54 between the acquisition layer and the absorbent core, having a shape as generally illustrated in FIGS. 1-6 as described above. The width of the rectangular acquisition layer 52 is 105 mm and the maximum width W3 of the distribution layer 54 in this example is 80 mm.

A diaper according to the above example was tested against size 4 reference diapers having a rectangular absorbent layer having a 110 mm width and a substantially rectangular distribution layer having a width of 80 mm (also including a rounded back edge as in the diaper example) and more SAP (NS L805, 13.2 g). The invention example 1 despite having less SAP had overall lower urine leakage compared to the reference (however without statistical certainty).

### Example 2

The diaper of example 2 was also constructed has generally illustrated in FIGS. 1-6 and had the following specifications:
W1=110 mm
W2=76 mm
W3=80 mm
W4=62 mm

| | |
|---|---|
| L (length of the article) | 478 mm |
| L′ (length of the absorption layer) | 360 mm |
| L612 (length of the core narrow portion) | 148 mm |
| Length of channel 26 (in y direction) | 227 mm |
| Width of channel 26 | 8 mm |
| L″ (length of the distribution layer) | 298 mm |
| L548 (length of the distribution layer narrow portion 548) | 176 mm |
| L31 (length of the distribution channels in y direction) | 173 mm |

The SAP amount in example 2 is 12.4 g of Nippon Shokubai L900. An auxiliary slot glue layer is disposed on the inner side of the top core wrap layer with an overall width of 119 mm (slots 1 mm with 1 mm gap in-between)

A diaper according to this example 2 was tested against size 4 reference diapers having a rectangular absorbent layer having a 110 mm width and a substantially rectangular distribution layer having a width of 80 mm including a rounded back edge. The reference diaper comprised more SAP (NS L900, 13.0 g). The leakage study conducted showed that example 2 according to the invention was not statistically worse by more than 1.1% in leakage (with 95% statistical significance) despite the significantly lower amount of SAP.

### Example 3

The following illustrates dimensions and material for an exemplary core comprising a pair of channel-forming areas and discontinuous absorbent sideline areas, similar to the core represented in FIG. 13. The following are exemplary dimension values for an absorbent core suitable for a “size 4” (“Midi”) diaper, which can be recommended for baby weighing between 7 kg and 18 kg.
L′=400 mm
L″=150 mm
Length of portion 616=140 mm
Length of portion 612=160 mm
Length of portion 614=100 mm
W1=110 mm
W2=76 mm
W3=80 mm
W4=68 mm
Number of islands 63 on each sideline area 61, 62=15
Diameter of each island 63=5-8 mm
Gap between each island 63=2-5 mm
Total area L′×Wmax=44,000 mm2
Combined area of material free area 62, 64 (including islands)=4.600 mm2 (representing 10.5% of L′×Wmax)
Channel 30 Length (in y direction)=227 mm
Channel 30 Width=8 mm
Minimum Distance Channel—material free side area 62, 64=20 mm
Minimum Distance Between Channels=20 mm

In this example, the core wrap comprised a top layer C-wrapped around a bottom layer with adhesive longitudinal seals (as in FIG. 5). The top layer had a width of 165 mm, the bottom layer had a width of 130 mm. The final core bag width was approximately 120 mm. The top layer was a 8 gsm water permeable propylene spunbonded nonwoven (from Fitesa Germany) and the bottom layer an hydrophobic 10 gsm nonwoven (from Fibertex). The core wrap was sealed by gluing at its font end and its back end. The absorbent material was SAP particles, L805, ex Nippon Shokubai Group, which were deposited for one half on the top layer and the other half on the bottom layer of the core wrap and immobilized by a microfibrous glue and an auxiliary slot glue layer on one side of the core wrap (as indicated above). The total amount of SAP was 11 g. The basis weight of the SAP in the central area is advantageously longitudinally profiled so that more absorbency is available towards the middle and the front of the core. The total length of the core wrap was about 10 mm longer in the front and in the back than the central absorbent material portion to provide for sandwich front and back core wrap end seals.

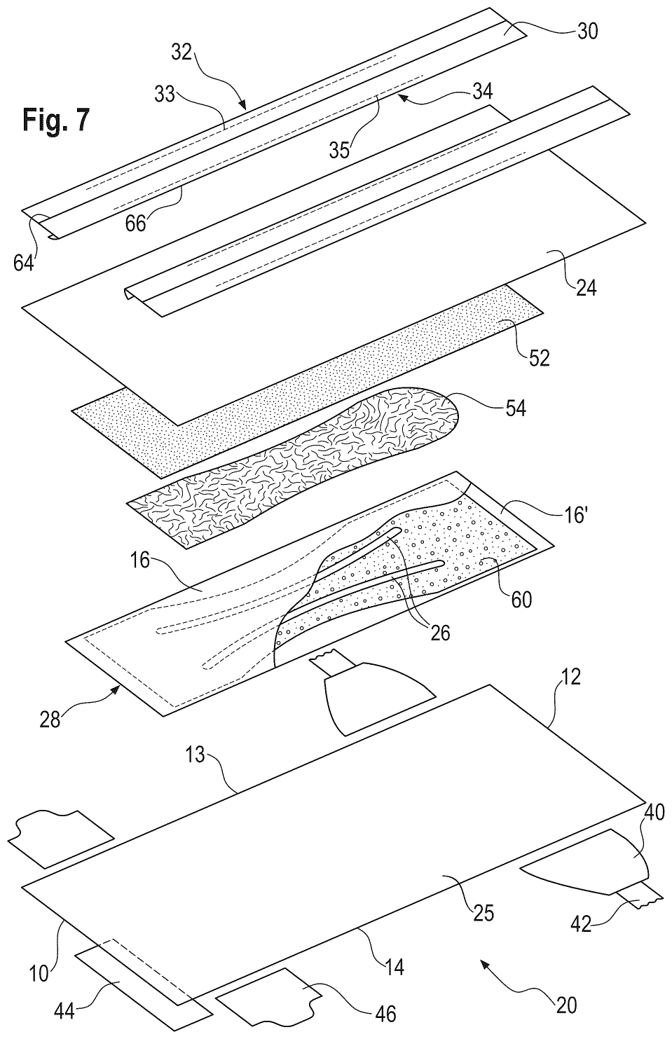

The absorbent core above was incorporated in a Size 4 diaper chassis similar to commercially available Pamper Swaddlers Size 4, comprising a dual acquisition distribution layer comprising a nonwoven acquisition layer 52 and a distribution layer 54 without distribution channels (as illustrated in FIG. 7) as described above. The width of the rectangular acquisition layer 52 was in this example 105 mm and the distribution layer 54 has a narrow central portion with a minimum crotch width (80-68-80 mm) and a rounded bullet end towards the back of the diaper.

As comparison, commercially available Pampers Swaddlers Size 4 having a rectangular absorbent material area including a pair of core channel. The core channels, the SAP amount and provenance, and the acquisition layer were as in the invention example. The distribution layer had constant width of 80 mm with a bullet shape towards the back of the diaper.

### Testing

Both diapers were put on an agile mannequin having a fluid inlet in the crotch allowing two gushes of 75 ml saline solution (total 150 ml) with 5 minutes of controlled simulated motion after each gush. The vertical position (sagging) and width of the crotch portion of the diaper were then measured. The measurements were repeated at least 5 times for each products.

### Results

The inventive diaper had a vertical position on average 5 mm higher than for the control marketed diapers. Similarly, the average width of the inventive diaper was 58 mm vs 68 mm for the marketed diapers. These results were statistically significantly different, and show that the inventive diapers have a better overall wet fit (less sagging and side expansion) than the control diapers.

### Packaging

The absorbent articles of the present disclosure may be placed into packages. The packages may comprise polymeric films and/or natural fibers and/or other materials. The package material may comprise paper, paper with a barrier layer, or a paper/film laminate. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages.

The absorbent articles may be packaged in any type of conventional packaging. The absorbent articles may be in particular compressed when packaged to save space. The package may thus comprise a plurality of bi-folded absorbent articles, wherein the articles in the package have an in-bag stack height of less than about 80 mm, according to the In-Bag Stack Height Test as described in WO2011/041352 (Weisman et al.), incorporated herein by reference. The packaged absorbent articles may for example have an in-bag stack height of from about 72 mm to about 80 mm or from about 74 mm to about 78 mm, specifically reciting all 0.5 mm increments within the specified ranges and all ranges formed therein or thereby, according to the In-Back Stack Height Test. The method used to measure the In-Bag Stack Height is described in further details in WO2011/041352 (Weisman) with the Universal Diaper Packaging Tester illustrated in FIG. 19 of WO2008/155702A1 (Hundorf).

Many absorbent articles are bi-folded along their transversal axis 90 when packed in their bags. When the articles are highly compressed in the bag to save space, this may cause a permanent fold line to appear along the bi-fold line of the articles, depending on the material used and the storage time of the articles in bag. Thus it is also considered that the articles may be packaged under a lower compression to avoid this issue, for example corresponding to an in-bag stack height above 80 mm, in particular between 84 mm and 120 mm. The articles may also be packaged tri-folded, as exemplarily disclosed in WO2008/155702 (Hundorf).

### Test Procedures

The values indicated herein are measured according to the methods indicated herein below, unless specified otherwise. All measurements are performed at 21° C.±2° C. and 50%±20% RH, unless specified otherwise. All samples should be kept at least 24 hours in these conditions to equilibrate before conducting the tests, unless indicated otherwise. All measurements should be reproduced on at least 4 samples and the average value obtained indicated, unless otherwise indicated.

### Centrifuge Retention Capacity (CRC)

The CRC measures the liquid absorbed by the superabsorbent polymer particles for free swelling in excess liquid. The CRC is measured according to EDANA method WSP 241.2-05.

### Urine Permeability Measurement (UPM) Test Method

This method is used to determine the permeability of a swollen hydrogel layer. The results are generally expressed in UPM units equal to 1×10-7 cm3·s/g. The Urine Permeability Measurement Test is disclosed in PCT application WO2012/174026A1, incorporated herein by reference.

### Thickness Measurement Method

This method is used to measure the thickness of a component of an article, or of the article (“sample”) itself in a standardized manner.

Equipment: Mitutoyo manual caliper gauge with a resolution of 0.01 mm, or equivalent instrument.

Contact Foot: Flat circular foot with a diameter of 17.0 mm (±0.2 mm). A circular weight may be applied to the foot (e.g., a weight with a slot to facilitate application around the instrument shaft) to achieve the target weight. The total weight of foot and added weight (including shaft) is selected to provide 4.14 kPa of pressure to the sample.

The caliper gauge is mounted with the lower surface of the contact foot in a horizontal plane so that the lower surface of the contact foot contacts the center of the flat horizontal upper surface of a base plate approximately 20 cm×25 cm. The gauge is set to read zero with the contact foot resting on the base plate.

Ruler: Calibrated metal ruler graduated in mm.

Stopwatch: Accuracy 1 second.

Sample preparation: The sample is conditioned at least 24 hours as indicated above.

Measurement procedure: The sample is laid flat with the bottom side, i.e. the side intended to be placed away from the wearer facing down. The point of measurement (if not otherwise indicated the middle of the sample) is carefully drawn on the top side of the sample, taking care not to compress or deform the sample.

The contact foot of the caliper gauge is raised and the sample is placed flat on the base plate of the caliper gauge with the top side of the sample up so that when lowered, the center of the foot is on the marked measuring point.

The foot is gently lowered onto the sample and released (ensure calibration to “0” prior to the start of the measurement). The caliper value is read to the nearest 0.01 mm, 10 seconds after the foot is released.

The procedure is repeated for each sample. Ten samples are measured in this manner for a given material and the average caliper is calculated and reported with an accuracy of one tenth mm.

### GENERAL

Dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal hygiene absorbent article comprising a longitudinal axis extending in a longitudinal direction, a transversal axis extending in a transversal direction, and a vertical direction orthogonal to the transversal direction and longitudinal direction, the article comprising, in this vertical order:
a liquid permeable topsheet;
a fibrous distribution layer having a front edge, a back edge and two longitudinal edges and a maximum width W3, wherein all width herein are measured in the transversal direction;
an absorbent core comprising a core wrap having a top layer and a bottom layer, an absorbent layer disposed between the core wrap top layer and the core wrap bottom layer, the absorbent layer comprising superabsorbent polymer particles, the absorbent layer having a front edge, a back edge and two longitudinal edges, the absorbent layer having a maximum width W1; and
a liquid-impermeable backsheet;
wherein the absorbent layer comprises a narrow portion at a position intermediate the front edge and the back edge of the absorbent layer, the narrow portion having a minimum width W2; wherein W1 is larger than W2 by a factor of at least 1.1;
the distribution layer comprises a narrow portion between the front edge and the back edge of the distribution layer, the narrow portion having a minimum width W4; wherein W3 is larger than W4 by a factor of at least 1.1;
wherein the narrow portion of the absorbent layer and the narrow portion of the distribution layer are at least partially vertically superposed;
wherein W1 is larger than or equal to W3, and W2 is larger than or equal to W4; and
wherein either the absorbent layer comprises at least one longitudinally-extending core channel or the distribution layer comprises at least longitudinally-extending distribution channel or both.

2. The absorbent article according to claim 1, wherein W1 is larger than W2 by a factor not exceeding 2.0.

3. The absorbent article according to claim 1, wherein W3 is larger than W4 by a factor not exceeding 2.0.

4. The absorbent article according to claim 1, wherein W1 is larger than W3 by at least 5 mm and W2 is larger than W4 by at least 5 mm.

5. The absorbent article according to claim 1, wherein the longitudinal edges of the absorbent layer and/or the distribution layer are inwardly curved in their respective narrow portion.

6. The absorbent article according to claim 1, wherein the part of the narrow portion of respectively the absorbent layer and distribution layer having the minimum width W2 and W4 is in the crotch region of the absorbent article, and wherein the crotch region is the central third of the article as measured in longitudinal direction.

7. The absorbent article according to claim 1, wherein the absorbent layer comprises one or more longitudinally-extending channel(s), and wherein the channel(s) is/are substantially free of absorbent material and the top layer of the core wrap is bonded to the bottom layer of the core wrap through at least one longitudinally-extending channel, so that a three-dimensional channel is formed when the absorbent material surrounding the channel swells.

8. The absorbent article according to claim 7, wherein the absorbent layer comprises at least two longitudinally-extending channels disposed symmetrically on each side of the longitudinal axis, and wherein the channels are at least partially disposed in the narrow portion of the absorbent layer and are inwardly curved towards the longitudinal axis.

9. The absorbent article according to claim 1, comprising an auxiliary glue between either the core wrap top layer and the absorbent layer or between the core wrap bottom layer and the absorbent layer or both.

10. The absorbent article according to claim 1, wherein the auxiliary glue is present at least in the narrow portion of the absorbent layer, and wherein the width of the auxiliary glue area where the auxiliary glue is present is larger than the absorbent layer's minimum width W2.

11. The absorbent article according to claim 1, wherein the absorbent material is free of cellulose fibers and the superabsorbent polymer particles are at least partially immobilized within the core wrap by a microfibrous glue.

12. The absorbent article according to claim 1, wherein the distribution layer comprises at least one distribution channel, and wherein the distribution channel is at least partially superposed with at least one channel of the absorbent core.

13. The absorbent article of claim 12, wherein the article comprises a pair of distribution channels at least partially vertically superposed with a pair of channels of the absorbent core.

14. The absorbent article according to claim 1, comprising an acquisition layer between the topsheet and the distribution layer, and wherein the distribution layer comprises or consists of cross-linked cellulose fibers.

15. The absorbent article according to claim 14, wherein an adhesive deposited on the acquisition layer at least partially immobilizes the fibers of the distribution layer.

16. The absorbent article according to claim 1, wherein the superabsorbent polymer is in the form of superabsorbent particles mixed with cellulose fibers.

17. The absorbent article according to claim 1, wherein the absorbent article comprises a pair of elasticized inner leg cuffs, each inner leg cuff having a proximal edge attached to the topsheet, and a free-standing elasticized distal edge;
the article comprises a pair of elasticized outer cuffs; and
wherein at least one of the elasticized inner cuffs and/or outer cuffs exerts a contraction force on the chassis of the article so that the absorbent article takes a basin shape when the article is placed on a wearer.

18. A personal hygiene absorbent article comprising a longitudinal axis extending in a longitudinal direction, a transversal axis extending in a transversal direction, and a vertical direction orthogonal to the transversal direction and longitudinal direction, the article comprising, in this vertical order:
a liquid permeable topsheet;
a fibrous distribution layer having a front edge, a back edge and two longitudinal edges and a maximum width W3;
an absorbent core comprising a core wrap having a top layer and a bottom layer, an absorbent layer disposed between the core wrap top layer and the core wrap bottom layer, the absorbent layer comprising superabsorbent polymer particles and cellulose fibers, the absorbent layer having a front edge, a back edge and two longitudinal edges, the absorbent layer having a maximum width W1;
a liquid-impermeable backsheet;
wherein the absorbent layer comprises a narrow portion at a position intermediate the front edge and the back edge of the absorbent layer, the narrow portion having a minimum width W2;
wherein W1 is larger than W2 by a factor of at least 1.1;
the distribution layer comprises a narrow portion between the front edge and the back edge of the distribution layer, the narrow portion having a minimum width W4; wherein W3 is larger than W4 by a factor of at least 1.1;
wherein the narrow portion of the absorbent layer and the narrow portion of the distribution layer are at least partially vertically superposed;
wherein W1 is larger than or equal to W3, and W2 is larger than or equal to W4;
wherein the absorbent layer comprises at least one longitudinally-extending core channel;
wherein the absorbent article comprises a pair of elasticized inner leg cuffs, each inner leg cuff having a proximal edge attached to the topsheet, and a free-standing elasticized distal edge;
the article comprises a pair of elasticized outer cuffs; and
wherein at least one of the elasticized inner cuffs and/or outer cuffs exerts a contraction force on the chassis of the article so that the absorbent article takes a basin shape when the article is placed on a wearer.

19. A personal hygiene absorbent article comprising a longitudinal axis extending in a longitudinal direction, a transversal axis extending in a transversal direction, and a vertical direction orthogonal to the transversal direction and longitudinal direction, the article comprising, in this vertical order:
a liquid permeable topsheet;
a fibrous distribution layer having a front edge, a back edge and two longitudinal edges and a maximum width W3, wherein all width herein are measured in the transversal direction;
an absorbent core comprising a core wrap having a top layer and a bottom layer, an absorbent layer disposed between the core wrap top layer and the core wrap bottom layer, the absorbent layer comprising superabsorbent polymer particles at least partially immobilized a microfibrous glue, the absorbent layer having a front edge, a back edge and two longitudinal edges, the absorbent layer having a maximum width W1;
a liquid-impermeable backsheet;
wherein the absorbent layer comprises a narrow portion at a position intermediate the front edge and the back edge of the absorbent layer, the narrow portion having a minimum width W2; wherein W1 is larger than W2 by a factor of at least 1.1;
the distribution layer comprises a narrow portion between the front edge and the back edge of the distribution layer, the narrow portion having a minimum width W4; wherein W3 is larger than W4 by a factor of at least 1.1;
wherein the narrow portion of the absorbent layer and the narrow portion of the distribution layer are at least partially vertically superposed;
wherein W1 is larger than or equal to W3, and W2 is larger than or equal to W4;
wherein either the absorbent layer comprises at least one longitudinally-extending core channel; and
wherein the article comprises an acquisition layer between the topsheet and the distribution layer, and the distribution layer comprises or consists of cross-linked cellulose fibers.

20. The absorbent article of claim 19, wherein the distribution layer comprises at least one distribution channel, and wherein the distribution channel is at least partially superposed with at least one channel of the absorbent core.
